(19) United States
(12) Patent Application Publication
(10) Pub. No.: US 2022/0233440 A1
(43) Pub. Date: Jul. 28, 2022

(54) NANO- OR MICRO-EMULSION COMPOSITIONS AND METHODS OF USE THEREOF

(71) Applicant: Nutraceutical Wellness Inc., New York, NY (US)

(72) Inventors: Giorgio DELL'ACQUA, Jersey City, NJ (US); Pamela SCOCA, Somers, NY (US); Roland PERALTA, Monroe, NJ (US)

(21) Appl. No.: 17/583,636

(22) Filed: Jan. 25, 2022

Related U.S. Application Data
(60) Provisional application No. 63/141,602, filed on Jan. 26, 2021.

(51) Int. Cl. A61K 9/107; A61K 36/71; A61K 47/14; A61K 45/06; A61K 47/44; A61K 36/53; A61K 36/534; A61K 47/24; A61K 47/46; A61K 8/06; A61K 8/92; A61P 17/14; A61Q 7/00

(52) U.S. Cl. CPC A61K 9/1075; A61K 36/71; A61K 47/14; A61K 45/06; A61K 47/44; A61K 36/53; A61Q 7/00; A61K 47/24; A61K 47/46; A61K 8/068; A61K 8/922; A61P 17/14; A61K 36/534

(57) ABSTRACT

The present disclosure provides all-natural nano- or micro-emulsions for the treatment of the skin, hair follicles, and related conditions, methods of using such nano- or micro-emulsions, and methods for preparing such nano- or micro-emulsions. The present disclosure relates to nano- or micro-emulsion formulations with improved bioavailability, time of onset, cellular permeability, and viscosity that can be suitable for topical, sublingual, oral, and/or nasal administrations. The current disclosure also includes compositions and methods for improving the bioavailability and the onset of actions arising from the particular components within the nano- or micro-emulsion formulations of the present disclosure.

### CROSS REFERENCE TO RELATED APPLICATIONS

This application claims priority to, and the benefit of, U.S. Provisional Application No. 63/141,602, filed on Jan. 26, 2021, the content of which is incorporated by reference in its entirety.

### BACKGROUND

Nano- or micro-emulsions are delivery systems that can be ingested or used topically to deliver active ingredients that are not otherwise bioavailable or cannot cross the skin barrier on their own. The components of a microemulsion may include an oil phase, an aqueous phase, a surfactant, and a cosurfactant. These emulsions can be oil in water (oil droplets in an aqueous phase), water in oil (water droplets in oil), or bicontinuous (one continuous phase). Nano- or micro-emulsions are of particular interest because of current and potential applications in fields such as pharmaceutical delivery, paints and coatings, food and beverage, and health and beauty aids.

The droplet size of emulsions are typically below 200 nm, although some have an upper limit of between 500 nm and 1000 nm. In order to achieve this droplet size and effectively deliver active ingredients, a high concentration of surfactant is usually required. Synthetic surfactants used at high concentration, such as sodium lauryl sulfate or polysorbates (e.g., Tween) are the gold standards for microemulsions; however, in topical or orally administered formulations, these synthetic surfactants can irritate the skin when applied topically and can damage the gastrointestinal (GI) tract when orally administered.

Hair loss, also known as alopecia, affects both males and females, and may be caused by genetic factors, auto-immune disorders, chemotherapy, aging, local or systemic disease, etc. None of the conditions causing alopecia are very well understood, however such conditions are generally distressing to the individual experiencing them, in part due to the human physical appearance often being regarded as an essential factor in social communication and interactions. Consequently, some individuals experiencing alopecia can perceive hair loss as a social handicap.

Nutraceutical formulations and the multi-targeting bioactive properties of certain plant phytonutrients offer a possible solution since they can target multiple triggers of hair loss at once. Further, the fact that these phytonutrients are natural in origin and known to be safe for consumption avoids many of the concerns of undesired side effects, which are common with pharmaceuticals. There is a significant need for compositions that avoid undesired side effects for use in reducing hair loss and/or facilitating hair growth or hair regrowth, after topical application to the skin. Some hair loss treatment formulations involve the use of synthetic vehicles/components, which may pose various problems and may lead to the irritation of the scalp or GI tract, depending on the route of administration. As a result, many people discontinue the use before seeing the potential beneficial effects from the hair loss treatment.

As such, there exists a need for improved all natural, non-synthetic, nano- and micro-emulsion compositions, methods of producing such emulsions and methods of using the same.

### SUMMARY

The present disclosure is directed to a nano- or micro-emulsion achieved using all-natural ingredients for the treatment of the skin and hair follicles of the eyelashes, beard, and scalp, among others. In addition, the nano- or micro-emulsion of the present disclosure is useful for the treatment of skin pigmentation or for the treatment of hair pigmentation. In some embodiments, the components of the nano- or micro-emulsion are active in the treatment of the skin and hair follicles as mentioned above and herein. In some embodiments, one or more nutraceuticals known to be advantageous for the treatment of one or more of the conditions described herein are comprised within the formulation of the present disclosure.

In some embodiments, the nano- or micro-emulsion comprises: (a) an oil dispersion comprising black cumin oil, and (b) an aqueous solution comprising a rhamnolipid. In some embodiments, the nano- or micro-emulsion comprises: (a) an oil dispersion comprising black cumin oil, and (b) an aqueous solution comprising a glycolipid. In some embodiments, the nano- or micro-emulsion comprises: (a) an oil dispersion comprising black cumin oil, and (b) an aqueous solution comprising a mixture of mono-rhamnolipids and di-rhamnolipids. In some embodiments, the nano- or micro-emulsion comprises: (a) an oil dispersion comprising black cumin oil, and (b) an aqueous solution comprising a mixture of rhamnolipids, wherein the mixture of rhamnolipids is 1:1 mono-rhamnolipid:di-rhamnolipid. In some embodiments, the nano- or micro-emulsion comprises: (a) an oil dispersion comprising black cumin oil, and (b) an aqueous solution comprising a mixture of rhamnolipids, wherein the mixture of rhamnolipids is 1:1.4 mono-rhamnolipid:di-rhamnolipid. In some embodiments, the nano- or micro-emulsion comprises: (a) an oil dispersion comprising black cumin oil, and (b) an aqueous solution comprising a mixture of glycolipids.

The present disclosure is directed to a method of providing a nano- or micro-emulsion. The method further provides a nano- or micro-emulsion for preventing hair loss, promoting hair growth and/or reducing hair loss by administering to a subject an effective amount of a composition as described herein, in a pharmaceutically suitable vehicle, for a time sufficient to promote hair regrowth and reduce hair loss in subjects.

In some embodiments, the micro- or nano-emulsion comprises (a) an oil dispersion comprising black cumin oil, and (b) an aqueous solution comprising a rhamnolipid. In some embodiments, the micro- or nano-emulsion comprises one or more of rosemary oil or peppermint oil. In some embodiments, the micro- or nano-emulsion comprises rosemary oil and peppermint oil. In some embodiments, the micro- or nano-emulsion comprises one or more of a mannosylerythritol lipid, phosphatidylcholine, and a preservative. In some embodiments, the micro- or nano-emulsion comprises a mannosylerythritol lipid, phosphatidylcholine, and a preservative. In some embodiments, the micro- or nano-emulsion comprises a preservative, wherein the preservative is one or more of Lactobacillus ferment and Lactobacillus Cocos Nucifera Fruit Extract ferment. In some embodiments, the micro- or nano-emulsion comprises a preservative and the preservative is a combination of Lactobacillus ferment and Lactobacillus Cocos Nucifera Fruit Extract ferment.

In some embodiments, the micro- or nano-emulsion comprises the black cumin oil in 0.1% wt/wt to 5% wt/wt. In some embodiments, the micro- or nano-emulsion comprises black cumin oil in about 1% wt/wt. In some embodiments, the micro- or nano-emulsion comprises the rhamnolipid in 1% wt/wt to 5% wt/wt. In some embodiments, the micro- or nano-emulsion comprises the rhamnolipid in about 1% wt/wt. In some embodiments, the micro- or nano-emulsion comprises rhamnolipid in about 2% wt/wt. In some embodiments, the micro- or nano-emulsion comprises rhamnolipid in about 5% wt/wt. In some embodiments, the micro- or nano-emulsion comprises black cumin oil and peppermint oil in a 1:1 ratio by weight. In some embodiments, the micro- or nano-emulsion comprises black cumin oil and rosemary oil in a 1:1 ratio by weight. In some embodiments, the micro- or nano-emulsion comprises black cumin oil, peppermint oil, and rosemary oil in a 1:1:1 ratio by weight.

In some embodiments, the micro- or nano-emulsion comprises 0.1-5% wt/wt black cumin oil; 0.1-5% wt/wt peppermint oil; and 0.1-5% wt/wt rosemary oil.

In some embodiments, the micro- or nano-emulsion comprises 0.1-5% wt/wt black cumin oil; 0.1-5% wt/wt peppermint oil: 0.1-5% wt/wt rosemary oil; and 0.1-1% wt/wt mannosylerythritol lipids.

In some embodiments, the micro- or nano-emulsion comprises 0.1-5% wt/wt black cumin oil; 0.1-5% wt/wt peppermint oil; 0.1-5% wt/wt rosemary oil; and 1-5% wt/wt rhamnolipids.

In some embodiments, the micro- or nano-emulsion comprises 0.1-5% wt/wt black cumin oil; 0.1-5% wt/wt peppermint oil: 0.1-5% wt/wt rosemary oil; and 0.1-2% wt/wt phosphatidylcholine.

In some embodiments, the micro- or nano-emulsion comprises (a) an oil dispersion comprising black cumin oil, and (b) an aqueous solution comprising a rhamnolipid wherein the oil dispersion particle size is less than 500 nm.

In some embodiments, the micro- or nano-emulsion comprises (a) an oil dispersion comprising black cumin oil, and (b) an aqueous solution comprising a rhamnolipid wherein the oil dispersion particle size is less than 500 nm for 90 days or more.

In some embodiments, the micro- or nano-emulsion comprises (a) an oil dispersion comprising black cumin oil, and (b) an aqueous solution comprising a rhamnolipid wherein the oil dispersion particle size is less than 500 nm for 6 months days or more.

In some embodiments, the micro- or nano-emulsion comprises (a) an oil dispersion comprising black cumin oil, and (b) an aqueous solution comprising a rhamnolipid wherein the oil dispersion particle size is less than 500 nm for 1 year or more.

In some embodiments, the micro- or nano-emulsion comprises (a) an oil dispersion comprising black cumin oil, and (b) an aqueous solution comprising a rhamnolipid wherein the oil dispersion particle size is less than 200 nm. In some embodiments, the micro- or nano-emulsion comprises (a) an oil dispersion comprising black cumin oil, and (b) an aqueous solution comprising a rhamnolipid wherein the oil dispersion particle size is less than 200 nm for 90 days or more.

In some embodiments, the micro- or nano-emulsion comprises (a) an oil dispersion comprising black cumin oil, and (b) an aqueous solution comprising a rhamnolipid wherein the oil dispersion polydispersity index (Pdi) less than 0.15. In some embodiments, the micro- or nano-emulsion comprises (a) an oil dispersion comprising black cumin oil, and (b) an aqueous solution comprising a rhamnolipid wherein the oil dispersion polydispersity index is less than 0.15 for 90 days or more.

In some embodiments, the micro- or nano-emulsion comprises (a) an oil dispersion comprising black cumin oil, and (b) an aqueous solution comprising a rhamnolipid wherein the oil dispersion polydispersity index (Pdi) less than 0.50. In some embodiments, the micro- or nano-emulsion comprises (a) an oil dispersion comprising black cumin oil, and (b) an aqueous solution comprising a rhamnolipid wherein the oil dispersion polydispersity index is less than 0.50 for 90 days or more. In some embodiments, the micro- or nano-emulsion comprises (a) an oil dispersion comprising black cumin oil, and (b) an aqueous solution comprising a rhamnolipid wherein the oil dispersion polydispersity index is less than 0.50 for 6 months or more. In some embodiments, the micro- or nano-emulsion comprises (a) an oil dispersion comprising black cumin oil, and (b) an aqueous solution comprising a rhamnolipid wherein the oil dispersion polydispersity index is less than 0.50 for 1 year or more.

In some embodiments, the micro- or nano-emulsion comprises (a) an oil dispersion comprising black cumin oil, and (b) an aqueous solution comprising a rhamnolipid wherein the oil dispersion polydispersity index (Pdi) less than 0.55. In some embodiments, the micro- or nano-emulsion comprises (a) an oil dispersion comprising black cumin oil, and (b) an aqueous solution comprising a rhamnolipid wherein the oil dispersion polydispersity index is less than 0.55 for 90 days or more. In some embodiments, the micro- or nano-emulsion comprises (a) an oil dispersion comprising black cumin oil, and (b) an aqueous solution comprising a rhamnolipid wherein the oil dispersion polydispersity index is less than 0.55 for 6 months or more. In some embodiments, the micro- or nano-emulsion comprises (a) an oil dispersion comprising black cumin oil, and (b) an aqueous solution comprising a rhamnolipid wherein the oil dispersion polydispersity index is less than 0.55 for 1 year or more.

In some embodiments, the micro- or nano-emulsion comprises (a) an oil dispersion comprising black cumin oil, and (b) an aqueous solution comprising a rhamnolipid wherein the composition comprises 80-95% wt/wt buffer solution and 0.1-10% wt/wt preservative

In some embodiments, the micro- or nano-emulsion comprises (a) an oil dispersion comprising black cumin oil, and (b) an aqueous solution comprising a rhamnolipid wherein the composition comprises 2% wt/wt Lactobacillus ferment; and 2% wt/wt Lactobacillus and Cocos Nucifera Fruit Extract.

In some embodiments, the micro- or nano-emulsion comprises an additional active pharmaceutical ingredient for the prevention of hair loss, reversing hair loss, and/or stimulating hair growth.

In some embodiments, the micro- or nano-emulsion is for use in the preparation of a medicament for the treatment of hair loss.

In some embodiments, the micro- or nano-emulsion is for use as a delivery vehicle for improving the bioavailability of other active ingredient(s).

In some embodiments, the application is directed to a method for preventing hair loss, reversing hair loss, treating hair loss, stopping hair loss, or stimulating hair growth in a subject, comprising administering the nano- or micro-emulsion composition of the present disclosure as discussed herein.

In some embodiments, the application is directed to a method for preventing hair loss, reversing hair loss, treating hair loss, stopping hair loss, or stimulating hair growth in a subject, comprising administering the nano- or micro-emulsion composition of the present disclosure as discussed herein wherein the nano- or micro-emulsion composition is topically administered or ingested.

In some embodiments, the application is directed to a method for preventing hair loss, reversing hair loss, treating hair loss, stopping hair loss, or stimulating hair growth in a subject, comprising administering the nano- or micro-emulsion composition of the present disclosure as discussed herein wherein the composition is administered topically.

In some embodiments, the application is directed to a method for improving bioavailability of an active pharmaceutical ingredient comprising adding the active pharmaceutical ingredient to the nano- or micro-emulsion of the present disclosure as discussed herein.

In some embodiments, the application is directed to a method for improving bioavailability of an active pharmaceutical ingredient comprising adding the active pharmaceutical ingredient to the nano- or micro-emulsion of the present disclosure as discussed herein wherein the nano- or micro-emulsion composition is topically administered or ingested.

In some embodiments, the application is directed to a method for improving bioavailability of an active pharmaceutical ingredient comprising adding the active pharmaceutical ingredient to the nano- or micro-emulsion of the present disclosure as discussed herein wherein the nano- or micro-emulsion composition is administered topically.

In some embodiments, the application is directed to a method for preparing a nano- or micro-emulsion composition for administration to a subject comprising:

(a) preparing an oil phase comprising black cumin oil;

(b) preparing an aqueous phase comprising a rhamnolipid:

(c) homogenizing the oil phase and the aqueous phase; and

(d) obtaining the nano- or micro-emulsion comprising an oil dispersion.

In some embodiments, the application is directed to a method for preparing a nano- or micro-emulsion composition for administration to a subject comprising:

(a) preparing an oil phase comprising black cumin oil;

(b) preparing an aqueous phase comprising a rhamnolipid;

(c) homogenizing the oil phase and the aqueous phase; and

(d) obtaining the nano- or micro-emulsion comprising an oil dispersion, and wherein the oil dispersion further comprises one or more of rosemary oil or peppermint oil.

In some embodiments, the application is directed to a method for preparing a nano- or micro-emulsion composition for administration to a subject wherein the aqueous phase comprises a buffered solution, wherein the pH is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, the application is directed to a method for preparing a nano- or micro-emulsion composition for administration to a subject wherein the aqueous phase comprises a buffered solution, wherein the pH is between about 6.0 and about 8.0. In some embodiments, the pH is between about 6.0 and between about 6.5. In some embodiments, the pH is between about 6.0 and between about 7.0. In some embodiments, the pH is between about 6.0 and between about 7.5. In some embodiments, the pH is between about 6.0 and between about 8.0. In some embodiments, the pH is between about 6.5 and between about 7.0. In some embodiments, the pH is between about 6.5 and between about 7.5. In some embodiments, the pH is between about 6.5 and between about 8.0. In some embodiments, the pH is between about 7.0 and between about 7.5. In some embodiments, the pH is between about 7.0 and between about 8.0. In some embodiments, the pH is between about 7.5 and between about 8.0.

In some embodiments, the application is directed to a method for preparing a nano- or micro-emulsion composition for administration to a subject wherein the method comprises a homogenizing step, wherein the homogenizing step comprises high shear homogenization. In some embodiments, the application is directed to a method for preparing a nano- or micro-emulsion composition for administration to a subject wherein the method comprises a homogenizing step, wherein the homogenizing step comprises high shear homogenization and high-pressure homogenization. In some embodiments, the application is directed to a method for preparing a nano- or micro-emulsion composition for administration to a subject wherein the method comprises a homogenizing step, wherein the homogenizing step comprises high shear homogenization followed by high-pressure homogenization. In some embodiments, the application is directed to a method for preparing a nano- or micro-emulsion composition for administration to a subject wherein the method comprises a homogenizing step, wherein the homogenizing step comprises performing high-pressure homogenization multiple times. In some embodiments, the present disclosure is directed to a method for preparing a nano- or micro-emulsion composition for administration to a subject wherein the method comprises a homogenizing step, wherein the homogenizing step comprises performing high-pressure homogenization at least two times. In some embodiments, the present disclosure is directed to a method for preparing a nano- or micro-emulsion composition for administration to a subject wherein the method comprises a homogenizing step, wherein the homogenizing step comprises performing high-pressure homogenization three times. In some embodiments, the present disclosure is directed to a method for preparing a nano- or micro-emulsion composition for administration to a subject wherein the method comprises a homogenizing step, wherein the homogenizing step comprises performing high-pressure homogenization at least three times.

The methods and corresponding compositions described herein are entirely natural microemulsion compositions. The methods and corresponding composition described herein are formed from plant-based ingredients. The methods and corresponding composition described herein are formed from plant-based ingredients and are non-toxic and non-irritating when compared to many synthetic alternatives. In some embodiments, the components in the natural microemulsion are biosurfactants. In some embodiments, the biosurfactants are naturally obtained from fermentation. ingredients and are non-toxic and non-irritating when compared to many synthetic alternatives. In some embodiments, the natural surfactants are safer and cause fewer side effects compared to synthetic surfactants. In some embodiments the biosurfactants are administered at a low dosage. In some embodiments, the components in the natural microemulsion are preservatives. In some embodiments, the preservatives are naturally obtained from fermentation.

Additional features, advantages, and aspects of the present disclosure are set forth or apparent from consideration of the following detailed description, drawings, and claims. Moreover, it is to be understood that both the foregoing summary of the present disclosure and the following detailed description are exemplary and intended to provide further explanation without limiting the scope of the present disclosure as claimed.

### BRIEF DESCRIPTION OF THE DRAWINGS

The accompanying drawings, which are included to provide a further understanding of the present disclosure, are incorporated in and constitute a part of this specification, illustrate aspects of the present disclosure and, together with the detailed description, serve to explain the principles of the present disclosure.

FIG. 1 depicts the interfacial tension results between oil and water with different oils and different surfactants in order to determine the most effective combinations.

FIG. 2A depicts the Z-average particle size of Formulation B at 30K PSI that was evaluated. The graph shows particle size (nm) over the course of one year at 4° C. and room temperature (RT).

FIG. 2B depicts the Z-average particle size of Formulation B at 20K PSI that was evaluated. The graph shows particle size (nm) over the course of one year at 4° C. and RT.

FIG. 2C depicts the polydispersity index (PDI) of the particle size of Formulation B at 20K PSI that was evaluated. The graph shows PDI of the particles in the formulation over the course of one year at 4° C. and RT

FIG. 2D depicts the polydispersity index (PDI) of the particle size of Formulation B at 30K PSI that was evaluated. The graph shows PDI of the particles in the formulation over the course of one year at 4° C. and RT

FIG. 3A depicts the particle size distributions for Formulation A, which was processed on a M110EH microfluidizer processor through the F12Y-H30Z interaction chamber at a pressure of 20,000 psi for 3 passes; measured using a Malvern zetasizer nano-s.

FIG. 3B depicts the particle size distributions for Formulation B, which was processed on a M110EH microfluidizer processor through the F12Y-H30Z interaction chamber at a pressure of 20,000 psi for 3 passes; measured using a Malvern zetasizer nano-s.

FIG. 4 depicts the processing conditions and particle size measurements (intensity based) for Formulations A and B, processed on the M110EH microfluidizer processor, measured using a Malvern zetasizer nano-s.

### DETAILED DESCRIPTION

The present disclosure is directed to a nano- or micro-emulsion. In some embodiments, the nano- or microemulsion is in the form of a composition. In some embodiments, the nano- or micro-emulsion composition comprises natural ingredients. In some embodiments, the nano- or micro-emulsion or compositions thereof, target stress related pathways. In some embodiments, the nano- or micro-emulsion comprises: (a) an oil dispersion comprising black cumin oil, and (b) an aqueous solution comprising a rhamnolipid.

The disclosure herein also provides a method of providing a nano- or micro-emulsion. The method further provides a nano- or micro-emulsion for preventing hair loss, promoting hair growth and/or reducing hair loss by administering to a subject an effective amount of the nano- or micro-emulsion. The disclosure herein provides a nano- or micro-emulsion for preventing hair loss, promoting hair growth and/or reducing hair loss by administering to a subject an effective amount of the nano- or micro-emulsion or a composition as described herein, in a pharmaceutically suitable vehicle, for a time sufficient to promote hair regrowth and reduce hair loss in subjects.

In some embodiments, the present disclosure is directed to a nano- or micro-emulsion comprising:

(a) an oil dispersion comprising black cumin oil, and

(b) an aqueous solution comprising a rhamnolipid.

In some embodiments, the present disclosure is directed to a composition comprising a nano- or micro-emulsion, the nano- or micro-emulsion comprising:

(a) an oil dispersion comprising black cumin oil, and

(b) an aqueous solution comprising a rhamnolipid.

In some embodiments, the present disclosure is directed to a nano- or micro-emulsion composition comprising:

(a) an oil dispersion comprising black cumin oil,

(b) an aqueous solution, and

(c) one or more surfactants comprising a rhamnolipid.

In some embodiments, the nano- or micro-emulsion or compositions thereof are used for treating a hair follicle. In some embodiments, the nano- or micro-emulsion or compositions thereof are used for treating a hair loss or for stimulating hair growth. In some embodiments, the nano- or micro-emulsion or compositions thereof are used for preventing loss of hair pigmentation. In some embodiments, the nano- or micro-emulsion or compositions thereof are used for treating loss of hair pigmentation. In some embodiments, the nano- or micro-emulsion or compositions thereof are used for stimulating hair re-pigmentation.

In some embodiments, the nano- or micro-emulsion or compositions thereof are used for treating the skin. In some embodiments, the nano- or micro-emulsion or compositions thereof are used for treating skin pigmentation.

In some embodiments, the present disclosure is directed to a method for preparing a nano- or micro-emulsion composition in a subject comprising:

(a) preparing an oil phase comprising black cumin oil;

(b) preparing an aqueous phase comprising a rhamnolipid;

(c) homogenizing the oil phase and the aqueous phase; and

(d) obtaining the nano- or micro-emulsion comprising an oil dispersion.

In some embodiments, the method for preparing a nano- or micro-emulsion is to prepare a medicament for treating a hair follicle.

In some embodiments, the method for preparing a nano- or micro-emulsion results in reduced particle sizes compared to other methods. In some embodiments, the method for preparing a nano- or micro-emulsion results in particle sizes that are more uniform compared to other methods. In some embodiments, the method for preparing a nano- or micro-emulsion results in more uniform size distribution of particle sizes compared to other methods.

The method and corresponding composition described herein is an entirely natural microemulsion. The components in the natural microemulsion are biosurfactants. In some embodiments, the biosurfactants are naturally obtained from fermentation. In some embodiments, the natural surfactants are safer and cause fewer side effects compared to synthetic surfactants. In some embodiments the biosurfactants are administered at a low dosage.

In some embodiments, the present disclosure relates to a nutraceutical composition for reducing hair loss and/or facilitating hair growth or hair regrowth, the composition comprising: (a) an oil dispersion comprising black cumin oil, and (b) an aqueous solution comprising a rhamnolipid.

In some embodiments, the present disclosure relates to a method of reducing hair loss and/or facilitating hair growth or hair regrowth comprising the steps of applying to skin of a mammal in need of such treatment a safe and effective amount of a nutraceutical composition comprising: (a) an oil dispersion comprising black cumin oil, and (b) an aqueous solution comprising a rhamnolipid.

In some embodiments, the surfactants of the present disclosure are biosurfactants. In some embodiments, the biosurfactants in the nano- or micro-emulsion includes one or more rhamnolipids. In some embodiments, the biosurfactants in the nano- or micro-emulsion includes one or more mannosylerythritol lipids (MELs). In some embodiments, the biosurfactants in the nano- or micro-emulsion comprises a high purity phosphatidylcholine. In some embodiments, the nano- or micro-emulsion comprises one or more biosurfactants. In some embodiments, the nano- or micro-emulsion comprises one or more rhamnolipid and one or more mannosylerythritol lipids.

In some embodiments, the micro- or nano-emulsion comprises black cumin oil, and rosemary oil as the oil phase. In some embodiments, the micro- or nano-emulsion comprises black cumin oil and peppermint oil as the oil phase. In some embodiments, the micro- or nano-emulsion comprises rosemary oil and peppermint oil as the oil phase. In some embodiments, the micro- or nano-emulsion comprises black cumin oil, rosemary oil and peppermint oil as the oil phase. This microemulsion is used to deliver these oils, as well as hydrophobic phytoactives for hair down the hair follicle. The microemulsion formula backbone is a functional product, with natural ingredients playing an active role in targeting hair follicle mechanisms.

The present disclosure finds application in all mammalian species, including both humans and animals. In humans, the compositions of the present disclosure can be applied for example, to the head, pubic area, upper lip, eyebrows, and eyelids. The methods can also be used for cosmetic reasons in animals, e.g. applied to the skin of dogs and cats having bald patches due to mange or other diseases.

Other aspects of the present disclosure will become readily apparent by those skilled in the art from the following detailed description, wherein it is shown and described only in the preferred embodiments, simply by way of illustration of the best mode. As will be realized, the disclosure is capable of other and different embodiments, and its several details are capable of modifications in various obvious respects, without departing from the spirit of the disclosure. Accordingly, the description is to be regarded as illustrative in nature and not as restricted.

### Black Cumin Oil

Black Cumin Oil is derived from the seeds of the Nigella sativa botanical, better known as the Fennel Flower. It is also commonly known by various other names, including Black Oil, Baraka, Fitch Oil, Kalajira Oil, Kalonji Oil, and Love in a Mist, to name only a few.

The thymoquinone-containing black cumin oil used to prepare the present disclosure may be obtained from any of the several different commercial sources (e.g. N.S. Oils, Kibbutz Sa'ad, Israel). Preferably, the oil is a cold pressed oil obtained from Nigella sativa seeds using a screw-driven oil press. It is to be noted that the species Nigella saliva is also known by a variety of common names including (but is not limited to) black cumin, black caraway, fennel, nigella, nutmeg flower, and roman coriander. It has been widely used as antihypertensive, liver tonics, diuretics, digestive, anti-diarrheal, appetite stimulant, analgesics, anti-bacterial and in skin disorders. Extensive studies on N. sativa have been carried out by various researchers and a wide spectrum of its pharmacological actions have been explored which may include antidiabetic, anticancer, immunomodulator, analgesic, antimicrobial, anti-inflammatory, spasmolytic, bronchodilator, hepato-protective, renal protective, gastro-protective, and antioxidant properties—all of which may be exploited by the present nano- or micro-emulsion and compositions thereof.

Black cumin oil contains many active components, such as thymoquinone (TQ), alkaloids (nigellicines and nigelledine), saponins (alpha-hederin), flavonoids, proteins, fatty acids, and many others. (Tavakkoli et al. J. Pharmacopuncture 2017 September; 20(3): 179-193). Black cumin oil also contains a good amount of various vitamins and minerals like Cu, P, Zn, and Fe. Many active compounds have been identified. (Srinivasan, Food Quality and Safety 2018, 1-16). Some compounds are thymoquinone (TQ) (˜30%-48%), thymohydroquinone, dithymoquinone (nigellone), p-cymene (˜7%-15%), carvacrol (˜6%-122%), 4-terpineol (˜2%-7%), t-anethole (˜1%-4%), sesquiterpene longifolene (˜1%-8%), α-pinene, and thymol. (Boskabady and Shirmohammadi, 2002; Ali and Blunden, 2003), but it also contains other compounds such as carvone, limonene, citronellol in trace amounts, and two varieties of alkaloids, i.e. isoquinoline alkaloids (e.g. nigellicimine and nigellicimine-N-oxide) and pyrazole alkaloids (e.g. nigellidine and nigellicine), α-hederin, a water soluble pentacyclic triterpene (Al-Jassir, 1992; Nickavar et al., 2003). Black cumin see oil also contains fatty oil rich in unsaturated fatty acids, constituting linoleic acid (50%-60/o), oleic acid (20%), eicosadienoic acid (3%), and dihomolinoleic acid (10/), and saturated fatty acids (palmitic and stearic acids) constitute up to 30%. α-Sitosterol is the major sterol, accounting for 44%-54% of the total sterols in the black cumin seed oils, followed by stigmasterol (6.57%-20.9% of total sterols) (Cheikh-Rouhou et al., 2008; Mehta et al., 2008).

Without being bound by any theory, the inventors herein discovered the surprising result that the black cumin oil contained in the present disclosure provided a surfactant-type properties to the nano- or micro-emulsion which allowed for a low level of overall surfactant (e.g., a low level of rhamnolipids) in the final nano- or micro-emulsion while still imparting superior properties.

### Mannosylerythritol Lipids

One important type of biosurfactant is mannosylerythritol lipids (MEL). MEL are glycolipids, with properties including, for example, viscosity reduction, emulsification, and nematode control. MEL and MEL-like substances are produced mainly by Pseudozyma spp., but some are also produced by Ustilago spp. (Arutchelvi et al., 2008).

As used, herein, a “MEL” or “mannosylerythritol lipid” refers to a biosurfactant comprising either 4-O-β-D-mannopyranosyl-meso-erythritol or 1-O-β-D-mannopyranosyl erythritol as the hydrophilic moiety, and fatty acid groups and/or acetyl groups as the hydrophobic moiety. Isomers and/or analogs thereof are also included. For example, MEL isomers can differ in bond type and bond location of the carbohydrate, fatty acid and/or acetyl groups.

“MEL” or “mannosylerythritol lipid” can also include MEL molecules that have been modified, either synthetically or in nature. For example, “MEL” or “mannosylerythritol lipid” can comprise different carbon-length chains or different numbers of acetyl and/or fatty acid groups. MEL and/or modified forms thereof according to the subject disclosure can include, for example, tri-acylated, di-acylated, mono-acylated, tri-acetylated, di-acetylated, mono-acetylated and non-acetylated MEL, as well as stereoisomers and/or constitutional isomers thereof. Furthermore, there can be one to three esterified fatty acids, from 6 to 12 carbons, or more, in chain length.

MEL can be produced in more than 93 different combinations that fall under 5 main categories: MEL A, MEL B, MEL D, Tri-acetylated MEL A, and Tri-acetylated MEL B/C. Current production techniques take 10 to 14 days for accumulation of MEL using P. aphidis.

The present disclosure comprises one or more biosurfactant selected from mannosylerythritol lipid A (MEL-A), mannosylerythritol lipid B (MEL-B), and mannosylerythritol lipid C (MEL-C).

Two principle forms of microbe cultivation exist for growing microbes and producing their growth by-products: submerged (liquid fermentation) and surface cultivation (solid-state fermentation (SSF)). Both cultivation methods require a nutrient medium for the growth of the microorganisms, and are classified based on the type of substrate used during fermentation (either a liquid or a solid substrate). The nutrient medium for both types of fermentation typically includes a carbon source, a nitrogen source, salts and other appropriate additional nutrients and microelements. MEL is a natural surfactant produced by yeast, and various physiological actions have been reported. Uses as external preparations and cosmetics are useful as anti-inflammatory agents and antiallergic agents, hair nourishing and hair-restoring agents, antibacterial action and surface tension reduction.

### Rhamnolipids

Rhamnolipids are glycolipids which are produced in free nature from certain bacteria, for example Pseudomonas aeruginosa. The microorganisms usually produce mixtures of rhamnolipids comprising mono- and di-rhamnolipids which have one or two rhamnose units per molecule and can contain lipid chains of different length. Rhamnolipids are small molecule natural products of microbial origin whose antibacterial and antifungal activities are well documented.

The structure diversity of rhamnolipids is determined by the number of rhamnose (one or two) and fatty acids (one or two), and the fatty acid composition. The length of the constituent fatty acids and their combinations has been found to be largely variable. To date, over 40 different rhamnolipid congeners have been described, though Rha-C10-C10 and Rha-Rha-C10-C10 are typically found to be the dominant components in a naturally occurring mixture. Rhamnolipids are commonly classified into two groups: monorhamnolipids and dirhamnolipids.

Rhamnolipids possess outstanding surfactant properties along with notable antimicrobial activities. As disclosed herein, when used as a biosurfactant, amphipathic rhamnolipids may dramatically increase the aqueous solubility, biomobility and bioavailability of poorly soluble substances, such as hydrophobic antibiotics and polycyclic aromatic hydrocarbons (PAHs).

Biosurfactants such as rhamnolipids are not petroleum-based, but they are bio-synthesized from natural organisms, and thus have the advantage of sustainability, biodegradability and low-toxicity. Rhamnolipid biosurfactants are classified as lipophilic.

An amphiphilic rhamnolipid molecule is composed of two moieties. One part is a hydrophilic sugar, mono- or di-rhamnose, and the other part is a hydrophobic lipid possessing one or two 3-hydroxy fatty acid residues. These residues may either be both fully saturated or one may be saturated and the other unsaturated, with either one or two double bonds. The lipid moiety is attached to the sugar by O-glycosidic linkage while the two 3-hydroxy acyl groups are joined together by the formation of an ester bond.

### Preservatives

Topical cosmetic, toiletry and pharmaceutical products such as creams, lotions, pastes, liquids, aerosols, shampoos, gels, wipes, sticks, powders and granules, are known in the art to be susceptible to microbial infestation. The raw materials, packaging, and manufacturing environment for these products are often not sufficiently sterile, such that small amounts of microbiological contaminants can enter into final products. In some embodiments, preservatives for compositions of this disclosure are Leucidal SF Max, Lactobacillus ferment, Lactobacillus Cocos Nucifera Fruit Extract, Lactobacillus/Cocos Nucifera Fruit Extract, Lactobacillus and Cocos Nucifera Fruit Extract, Lactobacillus Cocos Nucifera Fruit Extract, Glycerin, Leucidal® Liquid S F, Sharomix™ 705, Preservative ECO/Geogard® ECT, Geogard® 221 (aka. Cosgard), Rokonsal™ BSB-N, Euxyl® K 903, Naticide/Plantaserv Q and Dermorganics® 1388.

In some embodiments, preservatives that may be used with the present disclosure include but are not limited to alkyl esters of parahydroxybenzoic acid. In some embodiments, preservatives that may be used with the present disclosure include hydantoin derivatives such as 1,3-bis(hydroxymethyl)-5,5-dimethylhydantoin, propionate salts, and a variety of quaternary ammonium compounds such as benzalkonium chloride, quatemium 15 (Dowicil 200), benzethonium chloride, and methylbenzethonium chloride. In some embodiments, preservatives that may be used with the present disclosure include Disodium EDTA, phenoxyethanol, methyl paraben, propyl paraben, imidazolidinyl urea (commercially available as Germall 1157), sodium dehydroacetate or benzyl alcohol may be used with the present disclosure. In some embodiments, preservatives that may be used with the present disclosure include parabens, (e.g. Germaben II, Methylparben, Propylparaben, Butylparaben) which are economical and the most widely used group of preservatives; formaldahyde releasers (e.g. Germall Plus, DMDM Hydantoin, Imadozolidinyl Urea, Diazolidinyl Urea); isothiazolinones (e.g. Kathon); phenoxyethanol (e.g. Optiphen, Optiphen Plus (contains phenoxyethanol combined with others for broad spectrum protection)) which are often considered a “milder alternative” to traditional preservatives and are often combined with caprylyl glycol, sorbic acid/potassium sorbate or EDTA to create broad spectrum efficacy; organic acids (e.g. Benzoic Acid/Sodium Benzoate, Sorbic Acid/Potassium sorbate, Levulinic Acid, Anisic Acid) which are often combined with other organic acids or diazolidinyl urea (DU) for broad spectrum effectiveness.

The preservatives should be selected having regard for the use of the composition and possible incompatibilities between the preservatives and other ingredients in the emulsion. In some embodiments, preservatives are employed in amounts ranging from about 0% to about 10%, or from about 0.1% to about 5%, or from about 1% to about 4%, by weight of the composition. In some embodiments, one or more preservatives are employed in amounts ranging from about 0%, from about 0.1%, from about 0.5%, from about 1%, from about 2%, from about 3%, from about 4%, from about 5%, or from about 6% by weight of the composition.

### Definitions

While the following terms are believed to be well understood in the context used herein, the following definitions are set forth to facilitate explanation of the presently disclosed subject matter.

As used herein, “a”, “an”, and “the” refer to “one or more” when used herein, including the claims. Thus, for example, reference to “a carrier” includes mixtures of one or more carriers, two or more carriers, and the like.

As used herein, “comprising” means that other steps and other ingredients which do not affect the end result can be added. This term encompasses the terms “consisting of” and “consisting essentially of”. As used herein, the verb “comprise” as is used in this description and in the claims and its conjugations are used in its non-limiting sense to mean that items following the word are included, but items not specifically mentioned are not excluded.

As used herein, “mixtures” is meant to include a simple combination of materials and any compounds that may result from their combination.

As used herein, “molecular weight” or “M. Wt.” refers to the weight average molecular weight unless otherwise stated.

As used herein, the terms “include,” “includes,” and “including,” are meant to be non-limiting and are understood to mean “comprise,” “comprises,” and “comprising,” respectively.

As used herein, “percentage” or “%” refer to concentrations by weight or by mass, unless defined otherwise.

As used herein, “purify” or “purified” refer to freeing something of extraneous, contaminating or debasing matter. As used herein, a purified preparation of something refers to a preparation having a purity that is suitable for the present disclosure. Without wishing to be bound by theory, for example, a purified preparation of something refers to a preparation having a purity which includes but is not limited to at least 50%, at least 60%, at least 70%, at least 80%, at least 90%, or at least 99%.

As used herein, “sublingual,” means “under the tongue” and refers to a method of administering substances via the mouth in such a way that the substances are rapidly absorbed via the blood vessels under the tongue rather than via the digestive tract. In the present disclosure, the sublingual administration of the formulations as described herein refers to the actions of spraying the formulations under the tongue of a human subject.

As used herein, “intranasal” or “nasal” refers to a method of administering substances via the nasal cavity in such a way that the substances are rapidly absorbed via the blood vessels through the mucosal tissues in the nasal cavity. In the present disclosure, the intranasal administration of the formulations refers to the actions of spraying the formulations into the nasal cavity of a human subject. In other embodiments, the formulation can be administered via various routes.

As used herein, “mass,” “wt %”, “wt/wt,” or alternatively “weight,” mean the calculations of the mass of one or more components in a formulation divided by the total mass of the formulation. In some embodiments, the mass of each component and the total mass of the formulation can be determined by using analytical balances as is well known by those skilled in the art. In some embodiments, the mass or weight is determined on an as-is basis. In some embodiments, the calculations of the mass can include the mass of liquids present in the component and/or the formulation.

As used herein, the terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, “alopecia” refers to partial or complete hair loss on the scalp, including, but not limited to sparse hair growth, short hair growth, thin hair growth, etc. Hair loss also occurs in a variety of other conditions.

As used herein, the term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

“Eyebrow” as used in this document refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris such as dandruff and other small objects from falling into the eyes, as well as providing a more sensitive sense for detecting objects being near the eye, like small insects. Eyebrows also have an important facilitative function in communication, strengthening facial expressions such as surprise, confusion, or anger.

The terms “eyelash” and “lash” are used interchangeably to refer to one of the hairs that grow at the edge of the eyelid. Eyelashes protect the eye from debris and provide a warning that an object (such as an insect or dust mite) is near the eye (which then is closed reflexively).

As used herein, “leucidal SF max” refers to a natural preservative. In some embodiments, “leucidal SF max” refers to a natural preservative formed from fermentation, specifically a probiotic-based ingredient created by the fermentation of Lactobacillus in a defined growth medium.

As used herein, “AMTicide Coco” refers to a natural preservative. In some embodiments, “AMTicide Coco” refers to a natural preservative developed by fermenting Cocos mucifera (Coconut) fruit with Lactobacillus to deliver a non-irritating and effective product for extra protection against yeast and mold.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present disclosure. Generally the term “about”, as used herein when referring to a measurable value such as an amount of weight, time, dose, etc. is meant to encompass in one example variations of ±15% or 10%, in another example ±5%, in another example ±1%, and in yet another example ±0.1% from the specified amount, as such variations are appropriate to perform the disclosed method.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

While the disclosure has been described in connection with specific embodiments thereof, it will be understood that it is capable of further modifications and the present disclosure is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure. Where a list of values is provided, it is understood that ranges between any two values in the list are also contemplated as additional embodiments encompassed within the scope of the disclosure, and it is understood that each intervening value to the tenth of the unit of the lower limit unless the context clearly dictates otherwise, between the upper and lower limit of said range and any other listed or intervening value in said range is encompassed within the disclosure; that the upper and lower limits of said sub-ranges can independently be included in the sub-ranges is also encompassed within the disclosure, subject to any specifically excluded limit.

As used herein, a “carrier” is one or more components of the formulation that serve to transport, dissolve, or maintain other ingredients. In some embodiments, a carrier as used herein is an aqueous or hydrophilic carrier. In some embodiments, a carrier as used herein is an aqueous buffer. In some embodiments, the cosmetics carrier materials are solid or liquid at 25° C. (including highly viscous substances) as for example glycerol, 1,2-propylene glycol, 1,2-butylene glycol, 1,3-propylene glycol, 1,3-butylene glycol, ethanol, water and mixtures of two or more of said liquid carrier materials with water. The topical compositions of the present disclosure also comprise a dermatologically acceptable carrier for the composition. The phrase “dermatologically acceptable carrier”, as used herein, means that the carrier is suitable for topical application to the keratinous tissue, has good aesthetic properties, is compatible with the actives of the present disclosure and any other components, and will not cause any safety or toxicity concerns. A safe and effective amount of carrier is from about 50% to about 99.99%, preferably from about 60% to about 99.9%, more preferably from about 70% to about 98%, and even more preferably from about 80% to about 95% of the composition. In some embodiments, the present nano- or micro-emulsions composition is the carrier for another active ingredient. In some embodiments, the present nano- or micro-emulsions composition is the carrier for another active ingredient and improves stability. In some embodiments, the present nano- or micro-emulsions composition is the carrier for another active ingredient and improves bioavailability. In some embodiments, the present nano- or micro-emulsions composition is the carrier for another active ingredient and improves skin penetration. In some embodiments, the present nano- or micro-emulsions composition is the carrier for another active ingredient and improves skin penetration for the treatment of various skin disorders. In some embodiments, the present nano- or micro-emulsions composition is the carrier for another active ingredient and improves penetration of hair follices for the treatment of hair loss.

### Formulations/Compositions

In some embodiments, the present disclosure is directed to an oil-in-water nanoemulsion comprising, an oil dispersion comprising black cumin oil, and an aqueous solution comprising a rhamnolipid. In some embodiments, the present disclosure is directed to an oil-in-water nanoemulsion comprising black cumin oil, an aqueous solution, and a rhamnolipid. In some embodiments, the present disclosure is directed to an oil-in-water nanoemulsion comprising black cumin oil, an aqueous solution, a rhamnolipid, and one or more essential oil. In some embodiments, the essential oil is one or more of peppermint oil, rosemary oil, lavender oil, bergamot oil, lemon oil, orange, sandalwood oil, tea tree oil, chamomile oil, cedarwood oil, clary sage oil, lemongrass oil, carrot seed oil, and geranium oil. In some embodiments, the oil-in water nanoemulsion comprises an oil dispersion comprising black cumin oil, rosemary oil and peppermint oil, and an aqueous solution comprising a rhamnolipid and at least one additional surfactant. In some embodiments, the additional surfactant is naturally occurring. In some embodiments, the additional surfactant is produced by bio-fermentation.

In some embodiments, the present disclosure is directed to an oil-in-water microemulsion comprising, an oil dispersion comprising black cumin oil, and an aqueous solution comprising a rhamnolipid. In some embodiments, the oil-in water microemulsion comprises an oil dispersion comprising black cumin oil, rosemary oil and peppermint oil, and an aqueous solution comprising a rhamnolipid and at least one additional surfactant. In some embodiments, the additional surfactant is naturally occurring. In some embodiments, the additional surfactant is produced by bio-fermentation.

In some embodiments, the rosemary oil is replaced with one or more essential oils.

In some embodiments, the peppermint oil is replaced with one or more essential oils.

In some embodiments, both the rosemary oil and the peppermint oil is replaced with one or more essential oils. In some embodiments, the rosemary oil, peppermint oil, or both are replaced with one or more of lavender oil, bergamot oil, lemon oil, orange, sandalwood oil, tea tree oil, chamomile oil, cedarwood oil, clary sage oil, lemongrass oil, carrot seed oil, and geranium oil.

In some embodiments, any oil (individually or in aggregate) in the nano- or micro-emulsion formulations can be from about 0.1% to about 15% by mass, from about 0.1% to about 10% by mass, from about 0.1% to about 5% by mass, from about 0.1% to about 1% by mass, from about 0.5% to about 15% by mass, from about 0.5% to about 10% by mass, from about 0.5% to about 5% by mass, from about 0.5% to about 1.5% by mass, from about 0.5% to about 1% by mass, from about 1% to about 15% by mass, from about 1% to about 10% by mass, from about 1% to about 5% by mass inclusive of all ranges and subranges therebetween. In some embodiments, any oil in the nano- or micro-emulsion formulations are from about 0.5% to about 2.5% by mass. In some embodiments, any oil in the nano- or micro-emulsion formulations are about 1%. In some embodiments, any oil in the nano- or micro-emulsion formulations are about 0.5%.

In some embodiments, the nano- or micro-emulsion formulations as discussed herein comprise black cumin oil. In some embodiments, the black cumin oil in the nano- or micro-emulsion formulations can be from about 0.1% to about 15% by mass, from about 0.1% to about 10% by mass, from about 0.1% to about 5% by mass, from about 0.1% to about 1% by mass, from about 0.5% to about 15% by mass, from about 0.5% to about 10% by mass, from about 0.5% to about 5% by mass, from about 0.5% to about 1.5% by mass, from about 0.5% to about 1% by mass, from about 1% to about 15% by mass, from about 1% to about 10% by mass, from about 1% to about 5% by mass inclusive of all ranges and subranges therebetween. In some embodiments, any oil in the nano- or micro-emulsion formulations are from about 0.5% to about 2.5% by mass. In some embodiments, black cumin oil in the nano- or micro-emulsion formulations are about 1%. In some embodiments, black cumin oil in the nano- or micro-emulsion formulations are about 0.5%.

In some embodiments, the nano- or micro-emulsion formulations as discussed herein comprise rosemary oil. In some embodiments, the rosemary oil in the nano- or micro-emulsion formulations can be from about 0.1% to about 15% by mass, from about 0.2% to about 5% by mass, from about 0.3% to about 3% by mass, from about 0.4% to about 1% by mass, from about 0.5% to about 0.75% by mass, from about 0.3% to about 10% by mass, from about 5% to about 15% by mass, inclusive of all ranges and subranges therebetween. In some embodiments, any oil in the nano- or micro-emulsion formulations are from about 0.5% to about 1% by mass. In some embodiments, rosemary oil in the nano- or micro-emulsion formulations is about 0.5%. In some embodiments, rosemary oil in the nano- or micro-emulsion formulations is about 0.75%. In some embodiments, rosemary oil in the nano- or micro-emulsion formulations is about 1.0%.

In some embodiments, the nano- or micro-emulsion formulations as discussed herein comprise peppermint oil. In some embodiments, the peppermint oil in the nano- or micro-emulsion formulations can be from about 0.1% to about 15% by mass, from about 0.2% to about 5% by mass, from about 0.3% to about 3% by mass, from about 0.4% to about 1% by mass, from about 0.5% to about 0.75% by mass, from about 0.3% to about 10% by mass, from about 5% to about 15% by mass, inclusive of all ranges and subranges therebetween. In some embodiments, the peppermint oil in the nano- or micro-emulsion formulations are from about 0.5% to about 1% by mass. In some embodiments, the peppermint oil in the nano- or micro-emulsion formulations is about 0.5%. In some embodiments, the peppermint oil in the nano- or micro-emulsion formulations is about 0.75%. In some embodiments, the peppermint oil in the nano- or micro-emulsion formulations is about 1.0%.

In some embodiments, the ratio of components of the formulation is an important aspect of the present disclosure. In some embodiments, the black cumin oil and rosemary oil are in a 1:1 wt/wt ratio. In some embodiments, the black cumin oil and peppermint oil are in a 1:1 wt/wt ratio. In some embodiments, the peppermint oil and the rosemary oil are in a 1:1 wt/wt ratio. In some embodiments, the black cumin oil and rosemary oil are in a 2:1 wt/wt ratio. In some embodiments, the black cumin oil and peppermint oil are in a 2:1 wt/wt ratio. In some embodiments, the peppermint oil and the rosemary oil are in a 2:1 wt/wt ratio. In some embodiments, the black cumin oil and rosemary oil are in a 1:2 wt/wt ratio. In some embodiments, the black cumin oil and peppermint oil are in a 1:2 wt/wt ratio. In some embodiments, the peppermint oil and the rosemary oil are in a 1:2 wt/wt ratio.

In some embodiments, the black cumin oil and rosemary oil are in a 3:1 wt/wt ratio. In some embodiments, the black cumin oil and rosemary oil are in a 4:1 wt/wt ratio. In some embodiments, the black cumin oil and rosemary oil are in a 5:1 wt/wt ratio. In some embodiments, the black cumin oil and rosemary oil are in a 1:3 wt/wt ratio. In some embodiments, the black cumin oil and rosemary oil are in a 1:4 wt/wt ratio. In some embodiments, the black cumin oil and rosemary oil are in a 1:5 wt/wt ratio.

In some embodiments, the black cumin oil and peppermint oil are in a 3:1 wt/wt ratio. In some embodiments, the black cumin oil and peppermint oil are in a 4:1 wt/wt ratio. In some embodiments, the black cumin oil and peppermint oil are in a 5:1 wt/wt ratio. In some embodiments, the black cumin oil and peppermint oil are in a 1:3 wt/wt ratio. In some embodiments, the black cumin oil and peppermint oil are in a 1:4 wt/wt ratio. In some embodiments, the black cumin oil and peppermint oil are in a 1:5 wt/wt ratio.

In some embodiments, the peppermint oil and rosemary oil are in a 3:1 wt/wt ratio. In some embodiments, the peppermint oil and rosemary oil are in a 4:1 wt/wt ratio. In some embodiments, the peppermint oil and rosemary oil are in a 5:1 wt/wt ratio. In some embodiments, the peppermint oil and rosemary oil are in a 1:3 wt/wt ratio. In some embodiments, the peppermint oil and rosemary oil are in a 1:4 wt/wt ratio. In some embodiments, the peppermint oil and rosemary oil are in a 1:5 wt/wt ratio.

In some embodiments, the black cumin oil, rosemary oil, and peppermint oil are in a 1:1:1 wt/wt/wt ratio. In some embodiments, the black cumin oil, rosemary oil, and peppermint oil are in a 2:1:1 wt/wt/wt ratio. In some embodiments, the black cumin oil, rosemary oil, and peppermint oil are in a 1:2:1 wt/wt/wt ratio. In some embodiments, the black cumin oil, rosemary oil, and peppermint oil are in a 1:1:2 wt/wt/wt ratio. In some embodiments, the black cumin oil, rosemary oil, and peppermint oil are in a 2:2:1 wt/wt/wt ratio. In some embodiments, the black cumin oil, rosemary oil, and peppermint oil are in a 1:2:2 wt/wt/wt ratio. In some embodiments, the black cumin oil, rosemary oil, and peppermint oil are in a 2:1:2 wt/wt/wt ratio.

In some embodiments, the rosemary oil and/or peppermint oil according to any of the embodiments herein are replaced with one or more additional oils, selected from the group consisting of lavender oil, bergamot oil, lemon oil, orange, sandalwood oil, tea tree oil, chamomile oil, cedarwood oil, clary sage oil, lemongrass oil, carrot seed oil, and geranium oil.

In some embodiments, any buffer or buffered solution in the nano- or micro-emulsion formulations can be from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, from about 80% to about 90%, from about 85% to about 90%, inclusive of all ranges and subranges therebetween. In some embodiments, the formulation described herein comprise an aqueous buffer. In some embodiments, the formulation described herein comprise an aqueous phosphate buffer. In some embodiments, the formulation described herein comprise an aqueous sodium phosphate buffer. In some embodiments, the formulation described herein comprise an aqueous carbonate buffer. In some embodiments, the formulation described herein comprise an aqueous sodium carbonate buffer.

In some embodiments, any surfactant in the nano- or micro-emulsion formulations can be from about 5% to about 90%, from about 10% to about 80%, from about 15% to about 70%, from about 20% to about 60%, from about 30% to about 50%, from about 35% to about 45%, from about 25% to about 75%, from about 45% to about 80%, inclusive of all ranges and subranges therebetween. In some embodiments, the surfactant is a biosurfactant.

In some embodiments, the nano- or micro-emulsion formulations as discussed herein comprise rhamnolipids. In some embodiments, the rhamnolipids in the present nano- or micro-emulsion formulations can range from about 0.1% to about 20% by mass, from about 0.5% to about 15% by mass, from about 1% to about 10% by mass, from about 1.5% to about 5% by mass, from about 1% to about 15% by mass, from about 5% to about 10% by mass, from about 10% to about 20% by mass, inclusive of all ranges and subranges therebetween. In some embodiments, the rhamnolipids in the present nano- or micro-emulsion formulations ranges from about 0.1% to about 20% by mass, including about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 8%, about 9%, about 10%, about 15%, or about 20%, inclusive of all ranges and subranges therebetween. In some embodiments, the rhamnolipids in the present nano- or micro-emulsion formulations range from about 2% to about 10%.

In some embodiments, the rhamnolipids in the present nano- or micro-emulsion formulations is about 0.1%. In some embodiments, the rhamnolipids in the present nano- or micro-emulsion formulations is about 0.2%. In some embodiments, the rhamnolipids in the present nano- or micro-emulsion formulations is about 0.3%. In some embodiments, the rhamnolipids in the present nano- or micro-emulsion formulations is about 0.4%. In some embodiments, the rhamnolipids in the present nano- or micro-emulsion formulations is about 0.5%. In some embodiments, the rhamnolipids in the present nano- or micro-emulsion formulations is about 0.6%. In some embodiments, the rhamnolipids in the present nano- or micro-emulsion formulations is about 0.7%. In some embodiments, the rhamnolipids in the present nano- or micro-emulsion formulations is about 08%. In some embodiments, the rhamnolipids in the present nano- or micro-emulsion formulations is about 0.9%. In some embodiments, the rhamnolipids in the present nano- or micro-emulsion formulations is about 1.0%.

In some embodiments, the rhamnolipids in the present nano- or micro-emulsion formulation is between about 0.1% and about 1.0%, between about 0.2% and about 1.0%, between about 0.3% and about 1.0%, between about 0.4% and about 1.0%, between about 0.5% and about 1.0%, between about 0.6% and about 1.0%, between about 0.7% and about 1.0%, between about 0.8% and about 1.0%, between about 0.9% and about 1.0%.

In some embodiments, the rhamnolipids in the present nano- or micro-emulsion formulation is between about 0.1% and about 0.9%, between about 0.2% and about 0.9%, between about 0.3% and about 0.9%, between about 0.4% and about 0.9%, between about 0.5% and about 0.9%, between about 0.6% and about 0.9%, between about 0.7% and about 0.9%, between about 0.8% and about 0.9%.

In some embodiments, the rhamnolipids in the present nano- or micro-emulsion formulation is between about 0.1% and about 0.8%, between about 0.2% and about 0.8%, between about 0.3% and about 0.8%, between about 0.4% and about 0.8%, between about 0.5% and about 0.8%, between about 0.6% and about 0.8%, between about 0.7% and about 0.8%.

In some embodiments, the rhamnolipids in the present nano- or micro-emulsion formulation is between about 0.1% and 0.7%, between about 0.2% and about 0.7%, between about 0.3% and about 0.7%, between about 0.4% and about 0.7/o, between about 0.5% and about 0.7%, between about 0.6% and about 0.7%.

In some embodiments, the rhamnolipids in the present nano- or micro-emulsion formulation is between about 0.1% and about 0.6%, between about 0.2% and about 0.6%, between about 0.3% and about 0.6%, between about 0.4% and about 0.6%, between about 0.5% and about 0.6%.

In some embodiments, the rhamnolipids in the present nano- or micro-emulsion formulation is between about 0.1% and about 0.5%, between about 0.2% and about 0.5%, between about 0.3% and 0.5%, between about 0.4% and about 0.5%.

In some embodiments, the rhamnolipids in the present nano- or micro-emulsion formulation is between about 0.1% and about 0.4%, between about 0.2% and about 0.4%, between about 0.3% and about 0.4%.

In some embodiments, the rhamnolipids in the present nano- or micro-emulsion formulation is between about 0.1% and about 0.3%, between about 0.2% and about 0.3%.

In some embodiments, the rhamnolipids in the present nano- or micro-emulsion formulation is between about 0.1% and about 0.2%.

In some embodiments, the rhamnolipids are a mono-rhamnolipids. In some embodiments, the rhamnolipids are di-rhamnolipids. In some embodiments the rhamnolipids are a mixture of rhamnolipids. In some embodiments the rhamnolipids are a mixture of mono-rhamnolipids and di-rhamnolipids. In some embodiments the rhamnolipids are a 1:1 mixture of mono-rhamnolipids:di-rhamnolipids. In some embodiments the rhamnolipids are a 1:1.1 mixture of mono-rhamnolipids:di-rhamnolipids. In some embodiments the rhamnolipids are a 1:1.2 mixture of mono-rhamnolipids:di-rhamnolipids. In some embodiments the rhamnolipids are a 1:1.3 mixture of mono-rhamnolipids:di-rhamnolipids. In some embodiments the rhamnolipids are a 1:1.4 mixture of mono-rhamnolipids:di-rhamnolipids. In some embodiments the rhamnolipids are a 1:1.5 mixture of mono-rhamnolipids:di-rhamnolipids. In some embodiments the rhamnolipids are a 1:1.6 mixture of mono-rhamnolipids:di-rhamnolipids. In some embodiments the rhamnolipids are a 1:1.7 mixture of mono-rhamnolipids:di-rhamnolipids. In some embodiments the rhamnolipids are a 1:1.8 mixture of mono-rhamnolipids:di-rhamnolipids. In some embodiments the rhamnolipids are a 1:1.9 mixture of mono-rhamnolipids:di-rhamnolipids. In some embodiments the rhamnolipids are a 1:2 mixture of mono-rhamnolipids:di-rhamnolipids.

In some embodiments the rhamnolipids are a 1.1:1 mixture of mono-rhamnolipids:di-rhamnolipids. In some embodiments the rhamnolipids are a 1.2:1 mixture of mono-rhamnolipids:di-rhamnolipids.

In some embodiments the rhamnolipids are a 1.3:1 mixture of mono-rhamnolipids:di-rhamnolipids. In some embodiments the rhamnolipids are a 1.4:1 mixture of mono-rhamnolipids:di-rhamnolipids. In some embodiments the rhamnolipids are a 1.5:1 mixture of mono-rhamnolipids:di-rhamnolipids. In some embodiments the rhamnolipids are a 1.6:1 mixture of mono-rhamnolipids:di-rhamnolipids. In some embodiments the rhamnolipids are a 1.7:1 mixture of mono-rhamnolipids:di-rhamnolipids. In some embodiments the rhamnolipids are a 1.8:1 mixture of mono-rhamnolipids:di-rhamnolipids. In some embodiments the rhamnolipids are a 1.9:1 mixture of mono-rhamnolipids:di-rhamnolipids. In some embodiments the rhamnolipids are a 2:1 mixture of mono-rhamnolipids:di-rhamnolipids.

In some embodiments, the nano- or micro-emulsion formulations as discussed herein comprise glycolipids. In some embodiments, the glycolipids in the present nano- or micro-emulsion formulations can range from about 0.1% to about 20% by mass, from about 0.5% to about 15% by mass, from about 1% to about 10% by mass, from about 1.5% to about 5% by mass, from about 1% to about 15% by mass, from about 5% to about 10% by mass, from about 10% to about 20% by mass, inclusive of all ranges and subranges therebetween. In some embodiments, the glycolipids in the present nano- or micro-emulsion formulations ranges from about 0.1% to about 20% by mass, including about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 8%, about 9%, about 10%, about 15%, or about 20%, inclusive of all ranges and subranges therebetween. In some embodiments, the glycolipids in the present nano- or micro-emulsion formulations range from about 2% to about 10%. In some embodiments, the glycolipids in the present nano- or micro-emulsion formulations is about 5%.

In some embodiments, the nano- or micro-emulsion formulations as discussed herein comprise one or more mannosylerythritol lipid. In some embodiments, the mannosylerythritol lipid in the present nano- or micro-emulsion formulations can range from about 0.1% to about 2% by mass, from about 0.1% to about 1% by mass, from about 0.1% to about 0.8% by mass, from about 0.15% to about 0.5%, from about 0.2% to about 0.5% by mass, inclusive of all ranges and subranges therebetween. In some embodiments, the mannosylerythritol lipid in the present nano- or micro-emulsion formulations ranges from about 0.1% to about 2% by mass, including about 0.1%, about 0.15%, about 0.2% about 0.25%, about 0.3%, about 0.35%, about 0.4%, about 0.45%, about 0.5%, about 0.55%, about 0.6%, about 0.65%, about 0.7%, about 0.8%, about 0.9%, about 1.0%, about 1.5%, or about 2.0%, inclusive of all ranges and subranges therebetween. In some embodiments, the mannosylerythritol lipid in the present nano- or micro-emulsion formulations range from about 0.1% to about 1%. In some embodiments, the mannosylerythritol lipid in the present nano- or micro-emulsion formulations is about 0.25%.

In some embodiments, the nano- or micro-emulsion formulations as discussed herein comprise phosphatidylcholine. In some embodiments, the phosphatidylcholine in the present nano- or micro-emulsion formulations can range from about 0.1% to about 2% by mass, from about 0.1% to about 1% by mass, from about 0.1% to about 0.8% by mass, from about 0.2% to about 1%, from about 0.2% to about 0.8% by mass, inclusive of all ranges and subranges therebetween. In some embodiments, the phosphatidylcholine in the present nano- or micro-emulsion formulations ranges from about 0.1% to about 2% by mass, including about 0.1%, about 0.15%, about 0.2% about 0.25%, about 0.3%, about 0.35%, about 0.4%, about 0.45%, about 0.5%, about 0.55%, about 0.6%, about 0.625%, about 0.65%, about 0.675%, about 0.7%, about 0.8%, about 0.9%, about 1.0%, about 1.5%, or about 2.0%, inclusive of all ranges and subranges therebetween. In some embodiments, the phosphatidylcholine in the present nanoemulsion formulations range from about 0.1% to about 1%. In some embodiments, the phosphatidylcholine in the present nanoemulsion formulations is about 0.625%.

In some embodiments, the present disclosure also provides methods of improving the bioavailability of one or more active ingredients in a subject comprising administering to the subject the nano- or micro-emulsion formulations as described herein.

### Droplet/Particle Size

As used herein, the term “nanoemulsion”, “microemulsion”, “nano- or micro-emulsion” means an oil-in-water (o/w) emulsion with an average particle size ranging from about 1 nm to about 1000 nm. Specifically, the term “nano- or micro-emulsion” refers to an emulsion with nanoemulsion components or microemulsion components or a combination of both microemulsion and nanoemulsion components.

Without being bound to any theory, microemulsions and nanoemulsions, despite having practically the same composition, have aspects that may distinguish them apart from each other. Both formulations are composed of a water and an oil phase and both formulations contain surfactants. The dispersion of one phase into the other is allowed by surfactants, amphiphilic molecules that decrease interfacial tension between the two phases (Pavoni L et al., 2020). The particle size distribution of microemulsions and nanoemulsions can be overlapped, as both have values in the nano range. Several authors defined different size values, with upper limits fixed to 100, 200, or 500 nm, making the distinction by particle size unclear (Pavoni L et al., 2020). The parameter that can be used to determine the difference between microemulsions and nanoemulsions is the free energy of the system. Microemulsions are considered to be thermodynamically stable, while nanoemulsions are kinetically stable. Because the two separate phases of microemulsions are energetically favorable, they can be achieved spontaneously by mixing the oil, water, and surfactants phases (the latter in high amounts or by using synthetic surfactants with very low interfacial tension properties). However, external energy such as magnetic stirring is often used to form microemulsions. Nanoemulsions can only be achieved by the presence of external energy input such as high-pressure homogenization, microfluidization, or sonication, to exceed the energy gap between the separate phases (Pavoni L et al., 2020). Because microemulsions are more thermodynamically stable, there is typically optical transparency in formulations, which is usually not always present in nanoemulsions (Fonseca-Santos B et al., 2015). Additionally, microemulsions are considered to have a more homogenous particle size than nanoemulsions, which typically have a range of heterogeneously sized droplets (Fonseca-Santos B et al., 2015).

Without being bound to any theory, benefits of nanoemulsions are that they can load a higher amount of dispersed phase (in the case of the present disclosure, oil) in the presence of a lower amount of surfactant. The surfactant-to-oil ratio (SOR) is generally >2 in microemulsions, and typically between 1-2 in nanoemulsions. As a result, the lower amount of surfactant in nanoemulsions is favorable as it guarantees a better toxicological/safety profile in comparison to microemulsions. Additionally, because of the external energy used to form nanoemulsions, they can be formulated with a greater variety of surfactants, while microemulsions require ones that provide an ultra low interfacial tension, such as synthetics (Pavoni L et al., 2020).

In some embodiments, the particle sizes referred to herein represent D₁₀ values. In some embodiments, the particle sizes referred to herein represent D₅₀ values. In some embodiments, the particle sizes referred to herein represent D₉₀ values. The particle size referred to herein is a z-average is optionally measured by dynamic light scattering. In addition, by controlling the methods of producing the nano- or micro-emulsion of the oil dispersion particles, the average cross-sectional diameters of the droplets that are produced may be controlled in certain embodiments. Those of ordinary skill in the art will be able to determine the average cross-sectional diameter (or other characteristic dimension) of a plurality or series of droplets, for example, using laser light scattering, microscopic examination, or other known techniques. The average cross-sectional diameter of a single droplet, in a non-spherical droplet, is the diameter of a perfect sphere having the same volume as the non-spherical droplet. The average cross-sectional diameter of a droplet (and/or of a plurality or series of droplets) may be, for example, in some embodiments, the particle size is less than 500 nm. In some embodiments, the particle size is less than 400 nm. In some embodiments, the particle size is less than 300 nm. In some embodiments, the particle size is less than 250 nm. In some embodiments, the particle size is less than 200 nm. In some embodiments, the particle size is between 100 nm and 1,000 nm. In some embodiments, the particle size is between 200 nm and 1,000 nm. In some embodiments, the particle size is between 300 nm and 1,000 nm. In some embodiments, the particle size is between 400 nm and 1.00 nm. In some embodiments, the particle size is between 500 nm and 1,000 nm. In some embodiments, the particle size is between 600 nm and 1,000 nm. In some embodiments, the particle size is between 700 nm and 1,000 nm. In some embodiments, the particle size is between 800 nm and 1,000 nm. In some embodiments, the particle size is between 900 nm and 1.00 nm. In some embodiments, the particle size is between 100 nm and 900 nm. In some embodiments, the particle size is between 200 nm and 900 nm. In some embodiments, the particle size is between 300 nm and 900 nm. In some embodiments, the particle size is between 400 nm and 900 nm. In some embodiments, the particle size is between 500 nm and 900 nm. In some embodiments, the particle size is between 600 nm and 900 nm. In some embodiments, the particle size is between 700 nm and 900 nm. In some embodiments, the particle size is between 800 nm and 900 nm. In some embodiments, the particle size is between 100 nm and 800 nm. In some embodiments, the particle size is between 200 nm and 800 nm. In some embodiments, the particle size is between 300 nm and 800 nm. In some embodiments, the particle size is between 400 nm and 800 nm. In some embodiments, the particle size is between 500 nm and 800 nm. In some embodiments, the particle size is between 600 nm and 800 nm. In some embodiments, the particle size is between 700 nm and 800 nm. In some embodiments, the particle size is between 100 nm and 700 nm. In some embodiments, the particle size is between 200 nm and 700 nm. In some embodiments, the particle size is between 300 nm and 700 nm. In some embodiments, the particle size is between 400 nm and 700 nm. In some embodiments, the particle size is between 500 nm and 700 nm. In some embodiments, the particle size is between 600 nm and 700 nm. In some embodiments, the particle size is between 100 nm and 500 nm. In some embodiments, the particle size is between 200 nm and 500 nm. In some embodiments, the particle size is between 300 nm and 500 nm. In some embodiments, the particle size is between 400 nm and 500 nm. In some embodiments, the particle size is between 100 nm and 400 nm. In some embodiments, the particle size is between 200 nm and 400 nm. In some embodiments, the particle size is between 300 nm and 400 nm. In some embodiments, the particle size is between 100 nm and 300 nm. In some embodiments, the particle size is between 200 nm and 300 nm. In some embodiments, the particle size is between 100 nm and 200 nm.

Additionally, in some embodiments, the average cross-sectional diameter of a droplet (and/or of a plurality or series of droplets) may be, for example, less than about 1 micrometers, less than about 500 nanometers, less than about 200 nanometers, less than about 100 nanometers, less than about 75 nanometers, or less than about 50 micrometers. The average cross-sectional diameter may also be at least about 100 nanometers, at least about 200 micrometers, at least about 300 nanometers, or at least about 500 nanometers. In some embodiments, at least about 50%, at least about 75%, at least about 90%, at least about 95%, or at least about 99% of the droplets within a plurality of droplets has an average cross-sectional diameter within any of the ranges outlined herein.

The droplets may be of substantially the same shape and/or size (i.e., “monodisperse”), or of different shapes and/or sizes, depending on the particular application. In some cases, the droplets may have a homogenous distribution of cross-sectional diameters, i.e., the droplets may have a distribution of cross-sectional diameters such that no more than about 25%, about 10%, about 5%, about 3%, about 1%, about 0.03%, or about 0.01% of the droplets have an average diameter that is more than about 25%, about 10%, about 5%, about 3%, about 1%, about 0.03%, or about 0.01% different from the average cross-sectional diameter of the droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link et al., published as WO 2004/091763 on Oct. 28, 2004, incorporated herein by reference, and in other references as described below and/or incorporated herein by reference.

### Applications

The systems and methods described herein can be used in a plurality of applications. For example, fields in which the particles nano- or micro-emulsions described herein may be useful include, but are not limited to, food, beverage, health and beauty aids, cosmetics, paints and coatings, chemical separations, and drugs and drug delivery.

In some embodiments, the nano- or micro-emulsion of the present disclosure is all natural. In some embodiments, the nano- or micro-emulsion of the present disclosure is itself a functional composition suitable to use in various methods. In some embodiments, the micro- or nano-emulsion of the present disclosure comprises one or more of the following components for the respective uses according to the following table, each of which are incorporated herein by reference. In some embodiments, the nano- or micro-emulsion of the present disclosure is an antimicrobial. In some embodiments, the nano- or micro-emulsion of the present disclosure stimulates hair growth. In some embodiments, the nano- or micro-emulsion of the present disclosure stimulates dermal papilla cell growth. In some embodiments, the nano- or micro-emulsion of the present disclosure protects the skin from stress response. In some embodiments, the nano- or micro-emulsion of the present disclosure protects the skin from DHT. In some embodiments, the nano- or micro-emulsion of the present disclosure stimulates dermal papilla cell repair. In some embodiments, the nano- or micro-emulsion of the present disclosure is an immune modulator.

| | |
|---|---|
| Black Cumin Seed | Antimicrobial (Ahmad A et al., 2013), Stimulates Hair Growth (Saleem |
| Oil | U et al., 2017), Dermal Papilla Cells Growth, Protects from Stress |
| | (Dell' Acqua G et al., 2020) |
| Peppermint Oil | Antimicrobial (Winska et al., 2019), Stimulates Hair Growth (Panahi et |
| | al., 2015; Murata et al., 2013) |
| Rosemary Oil | Antimicrobial (Winska et al., 2019), Stimulates Hair Growth (Oh et al., |
| | 2014) |
| Mannosylerythritol | Antimicrobial, Stimulates Papilla Cells Growth, Repairing (Morita T et |
| Lipids | al., 2013; Coelho ACS et al., 2020) |
| Rhamnolipids | Antimicrobial, Immune Modulators (Chen J et al., 2017) |
| Phosphatidylcholine | Lipid replenishment, Differentiation (PARP) (Furse S et al., 2015) |

The nano- or micro-emulsions described herein can be used as a topical formulation on the scalp. The nano- or micro-emulsions described herein can be used as a topical formulation on the skin. The nano- or micro-emulsions described herein can be used as a topical formulation on the face. The nano- or micro-emulsions described herein will allow oils and phytoactives included in the formulation to deliver to the hair follicle and benefit the hair growth cycle. The nano- or micro-emulsions described herein microemulsion utilizes biosurfactants and natural ingredients that will prevent scalp irritation and may provide moisturizing properties to the scalp. The nano- or micro-emulsions described herein microemulsion contains fully natural ingredients and is believed to be the first natural microemulsion that will enter the cosmeceutical market. The nano- or micro-emulsions described herein believe this microemulsion will have other benefits to the scalp and hair, as a result of the biosurfactants it is formulated with and may have antimicrobial properties against harmful bacteria. The nano- or micro-emulsions described herein can ultimately be utilized to create a product that can be taken orally to improve bioavailability of phytoactive or pharmaceutically active ingredients.

For instance, a precise quantity of a fluid, drug, nutraceutical, pharmaceutical, or other agent can be contained by the nano- or micro-emulsion designed to release its contents under particular conditions. In some instances, cells can be contained within a droplet, and the cells can be stored and/or delivered, e.g., to a target medium, for example, within a subject. Other agents that can be contained within a particle of the nano- or micro-emulsion described herein and delivered to a target medium include, for example, biochemical species such as nucleic acids such as siRNA, mRNA, RNAi and DNA, proteins, peptides, or enzymes. Additional agents that can be contained within an emulsion include, but are not limited to, viral particles, colloidal particles, magnetic particles, nanoparticles, quantum dots, fragrances, proteins, indicators, dyes, fluorescent species, chemicals, or the like. The target medium may be any suitable medium, for example, water, saline, an aqueous medium, or the like.

In some embodiments, the disclosed formulations are administered to a subject. In some embodiments, the nano- or micro-emulsion formulations as described herein can be used for sublingual administration. In some embodiments, the formulation as described herein can be used for intranasal or nasal administration. In some embodiments, the formulation as described herein can be used for topical administration. In some embodiments, the formulation as described herein can be ingested or orally administered.

In some embodiments, the emulsions described herein are administered to a hair follicle. In some embodiments, the emulsions described herein are administered topically to a hair follicle. In some embodiments, the emulsions described herein are administered topically to the scalp to treat, prevent, stop the progression of, or reverse hair loss. In some embodiments, the emulsions described herein comprise an additional active ingredient according to the disclosure herein and is administered to a hair follicle. In some embodiments, the emulsions described herein comprise an additional active ingredient according to the disclosure herein and is administered topically to a hair follicle. In some embodiments, the emulsions described herein comprise an additional active ingredient according to the disclosure herein and is administered topically to the scalp to treat, prevent, stop the progression of, or reverse hair loss.

The nano- or micro-emulsion may be applied to skin and hair using any suitable treatment regime. The nano- or micro-emulsion may be applied at least once a week, such as at least every two days, or at least once each day. For example, application may be twice per day.

In general, treatment using the nano- or micro-emulsion described here may be continued indefinitely. Alternatively, the treatment may be repeated only for a limited period, e.g. several weeks or months. Treatment may then be repeated for a similar period at a later date.

Most commonly, the area of the skin to which the composition is applied will be the scalp, i.e., the composition will be used to combat hair loss on the user's head. Other areas may be suitable for application, for example to promote the growth of eyebrow hair, eyelashes, beard or facial hair regions, or skin. In addition to treating or preventing hair loss and/or promoting the growth of the hair, the methods and compositions described here may also improve the appearance of hairs to which the composition is applied, e.g. by thickening the hair and improving the luster, condition and manageability of the hair.

In some embodiments, a method for treating hair loss comprises topical application of the nano- or micro-emulsion or compositions thereof on the scalp or any other body area where hair growth or regrowth is desirable. The nano- or micro-emulsion or compositions thereof may be useful for treating hair loss by preventing or slowing hair loss and/or stimulating or increasing hair growth or regrowth. The nano- or micro-emulsion or compositions thereof described herein may be useful in a wide variety of finished products, including pharmaceutical products and cosmetic products. The nano- or micro-emulsion may be prepared, packaged, and labeled for modulation of hair growth or regrowth, and for diminishing the hair loss process.

In some embodiments, the nano- or micro-emulsion disclosed herein may be topically administered. In an embodiment, the nano- or micro-emulsion or composition thereof may be topically applied to an area to be treated, for example the scalp in humans, by spraying, dabbing, swabbing, rubbing, or combinations thereof.

In some embodiments, the nano- or micro-emulsion may be topically applied in the form of a scalp stimulator foam. In another embodiment, the nano- or micro-emulsion may be topically dispersed on the scalp in an aerosol form such as in a chlorofluorocarbon solvent, for delivery in spray form. The spray form may present some advantages including high loading, enhanced drug uptake, convenient application, and less matting the hair in the region of application. In such embodiments, the nano- or micro-emulsion may remain on the scalp for a period of time of about 1 week, alternatively about 1 day, alternatively about 12 h, alternatively about 4 h, alternatively about 1 h, alternatively about 30 min, alternatively about 5 min, or alternatively about 1 min. The nano- or micro-emulsion may be removed at any desired point in time by washing and/or rinsing the scalp.

In some embodiments, the nano- or micro-emulsion may be topically administered at least on a daily, and preferably a twice daily, basis for a period of time sufficient to bring about the desired level of improvement in modulation of hair growth or regrowth. For example, a user may topically administer the nano- or micro-emulsion directly to a balding area or other area where increased hair growth is desired by gently massaging the composition of the present disclosure into the desired area. This process may be repeated later the same day. In an embodiment, the nano- or micro-emulsion may be left on the scalp or other area where increased hair growth is desired between applications occurring on the same day or on different days. As will be appreciated by one skilled in the art with the help of this disclosure, when the nano- or micro-emulsion may be topically applied/administered periodically on a routine basis prior to, during, and subsequent to modulation of hair growth or regrowth. Generally, the nano- or micro-emulsion may be topically administered on a daily basis, although more frequent applications also may be used.

In some embodiments, the application nano- or micro-emulsion may continue for any suitable period of time. For example, within a few weeks to a few months of the initial application, a user may notice a reduction in hair loss and/or an increase in hair growth or regrowth. It should be appreciated that the frequency with which the nano- or micro-emulsion should be applied will vary depending on the desired effect. In particular, the degree of cosmetic enhancement might vary directly with the total amount of nano- or micro-emulsion used.

In some embodiments, disclosed herein is a method of treating a skin or a hair condition comprising administering a composition to dermal papilla cells of a subject, wherein the composition comprises a nano- or micro-emulsion of black cumin oil, rosemary oil and/or peppermint oil.

As will be appreciated by those of skill in the art with the help of this disclosure, other methods may be used to topically apply/administer the nano- or micro-emulsion described herein.

In an embodiment, a composition for the treatment of hair loss such as nano- or micro-emulsion may be advantageously used to diminish hair loss and/or promote hair growth and/or regrowth. For example, as disclosed herein, a composition for the treatment of hair loss such as the nano- or micro-emulsion may diminish and/or stop hair loss in a time period of from about 7 days to about 80 days, alternatively from about 10 days to about 28 days, or alternatively from about 14 days to about 21 days.

While not intending to be limited by theory, it is believed that the nano- or micro-emulsion and compositions thereof may advantageously regrow hair in a time period of from about 4 weeks to about 20 weeks, alternatively from about 6 weeks to about 16 weeks, or alternatively from about 8 weeks to about 12 weeks.

In some embodiments, the nano- or micro-emulsion and compositions thereof advantageously diminish and/or stop the hair loss on the scalp when nano- or micro-emulsion and compositions thereof are topically applied to the scalp.

In an embodiment, the nano- or micro-emulsion and compositions thereof may advantageously promote hair growth from dormant and/or injured hair follicles, e.g., nano- or micro-emulsion and compositions thereof may have a rejuvenating effect on the hair follicles. Additional advantages of the nano- or micro-emulsion and compositions thereof and methods of using same may be apparent to one of skill in the art viewing this disclosure.

Dosage of the nano- or micro-emulsion of the disclosure is dependent upon many factors including, but not limited to, the severity of the hair loss, the subject's age, general health and individual response to the compositions of the disclosure. Accordingly, dosages of the compositions can vary and be readily adjusted, depending on each subject's response.

We further describe an article of manufacture containing a topical dosage form prepared from nano- or micro-emulsion, packaged for retail distribution, in association with instructions advising the consumer how to use the product to promote hair growth.

The nano- or micro-emulsion may be used to manufacture preparations to promote hair growth in other mammals besides humans. For example, the nano- or micro-emulsion may be used with farm animals such as sheep, in which fur (hair) growth would exhibit an economic benefit.

The nano- or micro-emulsion may also be used to stimulate hair growth in companion animals such as dogs, cats, gerbils, etc. The dosages required to obtain this effect will fit within the guidelines described above. Likewise, the nano- or micro-emulsion may be administered using formulations typically used for veterinary applications, taking into account the type of animal being treated. Other applications of the nano- or micro-emulsion to promote hair growth will become readily apparent to one skilled in the art based upon the disclosure of this application and should be considered to be encompassed by the claims.

### Stability

As used herein, “stability” refers to a desirable property of the provided concentrates and liquid dilution compositions, for example, the ability of the provided concentrates and liquid dilution compositions to remain free from one or more changes over a period of time, for example, at least or longer than 1 day, 1 week, 1 month, 1 year, or more. For example, a concentrate or liquid dilution composition can be described as stable if it is formulated such that it remains free from oxidation or substantial oxidation over time, remains of consistent clarity over time, remains safe and/or desirable for human consumption over time, remains safe and/or desirable for topical human application over time, has a lack of precipitates forming over time, has a lack of ringing overtime, and/or does not exhibit any visible phase separation over a period of time. For example, the concentrates and liquid dilution compositions can be described as stable if they exhibit one or more of these described characteristics, over time, when kept at a particular temperature, for example, room temperature, e.g., at or about 25° C., slightly below room temperature, e.g., between or between about 19° C. and 25° C., at refrigerated temperatures, e.g., at or about 4° C., or at frozen temperatures, e.g., at or about −20° C. or lower.

In some embodiments, the nano- or micro-emulsion of the present disclosure is stable. In some embodiments, the nano- or micro-emulsion of the present disclosure provides stability to any additional active ingredients. In some embodiments, the nano- or micro-emulsion of the present disclosure provides stability to the phytochemicals within the formulation. In some embodiments, the nano- or micro-emulsion of the present disclosure provides shelf stability to the phytochemicals within the formulation. In some embodiments, the nano- or micro-emulsion of the present disclosure provides stability and the stability is thermostability. In some embodiments, the nano- or micro-emulsion of the present disclosure provides stability and the stability is thermal stability. In some embodiments, the nano- or micro-emulsion of the present disclosure provides stability and the stability is long-term kinetic stability. In some embodiments, the nano- or micro-emulsion of the present disclosure provides stability and affords stability at room temperature of over 1 year.

In some embodiments, the nano- or micro-emulsion of the present disclosure is stable for more than 1 month, more than 2 months, more than 3 months, more than 4 months, more than 5 months, more than 6 months, more than 7 months, more than 8 months, more than 9 months, more than 10 months, more than 11 months, more than 1 year, more than 2 years, more than 3, years, more than 4 years, or more than 5 years. In some embodiments, the stability of the nano- or micro-emulsion is determined by the particle size.

### Hair Growth

Methods of assessing promotion of hair growth are known in the art and are described below. A straightforward method for assessing improvement in hair growth is by taking a photograph of a test area of the skin before and after application of nano- or micro-emulsion composition. The skin may optionally be shaved for this purpose. A photograph is taken. The treatment is then applied. A second photograph is then taken. The increase in hair growth may be quantified by counting any combination of: (a) number of hairs appearing; (b) length of hair appearing; (c) thickness of hair appearing; (d) straightness of hair appearing; (e) area of hair growth. Where the skin is not shaved, the relevant measurements may be with regard to improvement in the measured parameters, i.e., number of new hairs, increase in length of hair, increase in thickness of hair, increase in straightness of hair and increase in area of hair growth.

For example, hair growth may be assessed in an individual. An individual to whom the composition is administered may display enhanced hair growth, as measured by any of the parameters described above, of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more. This may be compared to hair growth in an individual to which the composition is not administered. The enhanced hair growth may be assessed by the number of additional or the number of thick or the number of straight hairs. Otherwise, it may be assessed by the thickness of hair growth. It may be assessed by an increased area of hair growth.

The nano- or micro-emulsion may be used to alleviate any type of alopecia, including androgenic alopecia and non-androgenic alopecia. Examples of non-androgenic alopecia include alopecia areata, alopecia due to radiotherapy or chemotherapy, scarring alopecia, stress related alopecia, etc. As used in this application, “alopecia” refers to partial or complete hair loss on the scalp, including, but not limited to sparse hair growth, short hair growth, thin hair growth, etc. Hair loss also occurs in a variety of in other conditions.

Anagen effluvium, is hair loss due to chemicals or radiation, such as chemotherapy or radiation treatment for cancer. It is also commonly referred to as “drug induced” or “radiation induced” alopecia. The nano- or micro-emulsion may used be to manufacture preparations to treat these types of alopecia.

Alopecia areata is an autoimmune disorder which initially presents with hair loss in a rounded patch on the scalp. It can progress to the loss of all scalp hair, which is known as alopecia totalis and to the loss of all scalp and body hair, which is known as alopecia universalis. The nano- or micro-emulsion may be used to manufacture preparations to treat these types of alopecia.

Traumatic alopecia is the result of injury to the hair follicle. It is also commonly referred to as “scarring alopecia”. Psychogenic alopecia occurs due to acute emotional stress. By inducing anagen, the nano- or micro-emulsion can be beneficial in these types of alopecia as well. Thus, the uses of the nano- or micro-emulsion are not limited to treating androgenetic alopecia. The nano- or micro-emulsion can be used to manufacture preparations to alleviate any type of hair loss.

Thus, the nano- or micro-emulsion can be applied topically to the scalp and hair to prevent or alleviate balding. Further, the nano- or micro-emulsion can be applied topically in order to induce or promote the growth of hair on the scalp.

The method of the disclosure can be used to stimulate hair growth or prevent hair loss in any situation where additional hair growth is desired. In particular, the method of the present disclosure is useful when a subject has experienced hair loss associated with various conditions, including but not limited to: anagen effluvium, drug properties Alopecia, radiation therapy, poisoning, diffuse alopecia areata, alopecia areata, loose anagen syndrome, postoperative occipital alopecia, syphilis, traction alopecia (traction alopecia), tricholtillomania tinea capitis, resting hair loss, telogen gravidarum, chronic resting hair loss, early male onset alopecia, iron deficiency, malnutrition/dyspepsia, Hypothyroidism, hyperthyroidism, systemic lupus erythematosus, chronic renal failure, liver dysfunction, advanced malignancy, viral or bacterial infection, and male developmental alopecia. In particular, the methods of the present disclosure are useful for male developmental alopecia, alopecia areata, alopecia in drug-induced alopecia (e.g. following cancer chemotherapy), and recovery of alopecia resulting from radiation therapy.

### Hair Pigmentation

Melanocytes present in the epidermis, in the bulb of the hair follicle and in the external root sheath of the hair follicle are mutually different. The major differences lie in the respective melanocyte-keratinocyte functional units. The melanin unit of the hair bulb is found in the bulb in the proximal anagen, which is an immunologically distinct region of the skin. Said unit comprises one melanocyte every 5 keratinocytes in the hair bulb, and one melanocyte every keratinocyte in the basal layer of the hair bulb matrix. Conversely, each epidermal melanocyte is associated with vital keratinocytes in the immunocompetent epidermial melanin unit.

The most evident difference between these two melanocyte populations is that the activity of the melanocyte in the hair bulb is subjected to cycle control and, therefore, the corresponding melanogenesis is strictly associated with the growth cycle of hair and is, hence, discontinuous. Epidermal melanogenesis, instead, appears to be continuous.

In fact, the hair cycle includes periods of melanocyte proliferation (during the early anagen phase), maturation (from halfway through to the end of the anagen phase), and death of melanocytes by apoptosis (during the early catagen phase). Every hair cycle is associated with the reconstruction of a pigment unit that is intact at least for the first ten cycles (Tobin, Int. J. Cosmetic Science, 2008; Tobin and Paus, Exp. Gerontol., 2001). Biosynthesis of melanin and its subsequent transfer from melanocytes to keratinocytes in the hair bulb depend on the availability of melanin precursors and on complex signal transduction mechanisms.

Though follicular and epidermal melanocytes have common traits, follicular melanocytes seem to be more sensitive than epidermal ones to the aging process. The pigmentary unit of hair plays an important role as environmental sensor, and also an important physiological function. In practice, pigments contribute to the is rapid excretion of heavy metals and toxins from the body through their selective bond with melanin (Tobin, Int. J. Cosmetic Science, 2008).

When grey and white hair appear, they suggest age-related and genetically regulated exhaustion of the pigment-forming potential of each hair follicle. The aging of melanocytes can be associated ‘with damage mediated by reactive oxygen species to the nucleus and to mitochondrial DNA with subsequent buildup of mutations with age, besides an evident alteration in antioxidant mechanisms or in pro-apoptotic and anti-apoptotic factors in cells. Oxidative stress is generated by several factors, such as environmental factors and endogenous changes (radiations, inflammation, emotional stress) that accelerate the aging process.

Other data in the literature report that the continuous synthesis of melanin during the growth phases of hair (anagen) generates high levels of oxidative stress, and that melanocytes are particularly sensitive to aging induced by free radicals. In fact, it has been proven that the pigmentary unit of grey hair contains apoptotic melanocytes and also presents a high level of oxidative stress.

In some embodiments, the present disclosure is directed to a method for effecting changes in mammalian hair appearance, hair growth, hair pigmentation and hair follicle and hair shaft size, comprising topical application to the skin of a mammal an effective amount of a topically active composition comprising the nano- or micro-emulsions of the present disclosure. In some embodiments all the methods and compositions herein are useful for the reduction of grey and white hair.

In some embodiments, the present disclosure is directed to a method for producing a melanogenetic action in the hair and to promote its pigmentation and pigmentation of the stem, comprising the step of administering to a subject in need thereof an effective amount of a composition comprising the nano- or micro-emulsions of the present disclosure.

### Skin Treatment

The compositions here may be useful for preventing, retarding, and/or treating uneven skin texture by regulating oily/shiny appearance, and regulating and/or reducing pore size appearance. The disclosure further relates to methods for regulating the condition of mammalian keratinous tissue wherein the methods each comprise the step of topically applying to the keratinous tissue of a mammal needing such treatment, a safe and effective amount of the skin care composition of the disclosure. In some embodiments, the nano- or micro-emulsions and compositions thereof described herein are effective for the treatment of pruritus, chronic pruritus, skin roughening, skin dryness, scar therapy, scar lightening, reduction of pathological myofibroblasts.

In some embodiments, the composition described herein is useful to treat chronic pruritus is defined as an itch persisting for >6 weeks, which can be severe enough to interfere with lifestyle activities.1 Pruritus can be a hallmark of many skin diseases as well as other non-cutaneous diseases. Neuropathic, psychogenic, systemic, and dermatologic disorders constitute the majority of causes of pruritus.

In some embodiments, the composition described herein is useful to treat skin roughening, mainly due to dryness, is generally caused by damage to the intracellular lipids of the skin, which decreases the water-retention capacity of the stratum corneum.

In some embodiments, the composition described herein is useful to treat scar therapy via the mechanism of targeted killing the myofibroblasts.

The composition may include disinfectants, antiseptics, or drug substances. Incorporation of one or more disinfectants or antiseptics is especially useful in those situations where it is important to inactivate the microorganisms which remain on the skin after normal cleansing. Incorporation of a drug substance in the composition may be useful for the prevention or treatment of various skin disorders or to deliver drug substances to the skin which are advantageously administered topically for percutaneous absorption.

In some embodiments, the composition described herein is useful to treat dermatosis. As used herein, term “dermatosis” should refer to the disease of skin, imbalance or defective, this includes but not limited to acne (including but not limited to acne vulgaris and acne rosacea), psoriasis, infect, flaw, pigmentation (include but not limited to inflammation after pigmentation (PIH)), hypopigmentation, hair growth imbalance (as the undue or unnecessary growth of alopecia and hair), pachylosis, skin is done, cutis laxa (include but not limited to skin-tightening and lack flexibility), wrinkle (including but not limited to microgroove and years stricture of vagina), blood vessel hyperplasia skin (including but not limited to skin dark stain), sebum generates imbalance (for example skin glow), the pore hypertrophy, excessively perspire (comprising hyperhidrosis), tatoo, erythra (comprising allergic rash and diaper rash), cicatrix, pain, scratch where it itches, burn, inflammation, wart, clavus, callus, edema, Rhus toxicodendron/poison lacquer rattan peel rash, skin carcinoma and insecticide, Aranea, biting of Serpentis and other animals.

In some embodiments, the composition described herein is useful to treat skin infections which includes but is not limited to acne, pustule, folliculitis, furunculosis, ecthyma, eczema, psoriasis, atoipc dermatitis, epidennolysis bullosa, ichthyosis, infected wound (ulcer that has for example infected, slight burns, incised wound, scratch, laceration, wound, tissue biopsy position, operative incision and sting place), herpes (for example cold sore) or other antibacterial or viral infection.

In some embodiments, the composition described herein is useful to treat wrinkles or skin lines which includes but is not limited to microgroove, deep wrinkle, laugh line, crows-feet, striae gravidarum, and liparitosis.

In some embodiments, the composition described herein is useful to treat variable color skin which includes but is not limited to pigmentation skin, hypopigmentation's skin, flaw skin, injury with blood-stasis and blood vessel hyperplasia skin.

In some embodiments, the composition described herein is useful to treat pigmentation of the skin which includes but is not limited to pigmentation (PIH) and other variable color skin after freckle, senile plaque (Exposure to Sunlight freckle), sunshine speckle, chloasma, the sick Huang of face, pigmentation, the inflammation. An example of hypopigmentation includes but is not limited to vitiligo.

In some embodiments, the composition described herein is useful to treat skin defects which includes but is not limited to the rash of pustule, blackhead, pimple, blackhead or other types relevant with acne. In some embodiments, the composition described herein is useful to treat dermopathic examples of cicatrix includes but is not limited to the cicatrix that caused by acne, operation, sting, burn, injured, wound and other wounds. In some embodiments, the composition described herein also can be used for the treatment of mucosal disease (for example oral cavity and vaginal mucosa disease). Include but is not limited to the example of mucosal disease periodontal disease, gingival, oropharynx cancer, candida mycoderma infect, cause such as herpes of mouth such as cold sore and fever blister and as herpes simplex or other viral infection of the genital herpes of genital ulcer.

### Additional Ingredients

In some embodiments, the composition described herein may optionally comprise one or more additional components known for use in skin care, hair care or personal care products, provided that the additional components are physically and chemically compatible with the essential components described herein, or do not otherwise unduly impair product stability, aesthetics or performance. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the conditioning composition.

In some embodiments, there is an optional hair growth stimulant component to the nano- or micro-emulsion of the present disclosure. The optional hair growth stimulant includes one or more of benzalkonium chloride, benzethonium chloride, phenol, estradiol, chlorpheniramine maleate, chlorophyllin derivatives, cholesterol, salicylic acid, cysteine, methionine, red pepper tincture, benzyl nicotinate, D,L-menthol, peppermint oil, calcium pantothenate, panthenol, castor oil, prednisolone, resorcinol, chemical activators of protein kinase C, glycosaminoglycan chain cellular uptake inhibitors, inhibitors of glycosidase activity, glycosaminoglycanase inhibitors, esters of pyroglutarylic acid, hexosaccharic acids or acylated hexosaccharic acids, aryl-substituted ethylenes, N-acylated amino acids, flavinoids, ascomycin derivatives and analogs, histamine antagonists such as diphenhydramine hydrochloride, triterpenes such as oleanolic acid and ursolic acid and those described in U.S. Pat. Nos. 5,529,769, 5,468,888, 5,631,282, and 5,679,705, JP 10017431, WO 95/35103, JP 09067253, WO 92/09262, JP 62093215, and JP 08193094; saponins such as those described in EP 0,558,509 to Bonte et al., and WO 97/01346 to Bonte et al, proteoglycanase or glycosaminoglycanase inhibitors such as those described in U.S. Pat. Nos. 5,015,470, 5,300,284, and 5,185,325, estrogen agonists and antagonists, pseudoterins, cytokine and growth factor promoters, analogs or inhibitors such as interleukin1 inhibitors, interleukin-6 inhibitors, interleukin-10 promoters, and tumor necrosis factor inhibitors, vitamins such as vitamin D analogs and parathyroid hormone antagonists, Vitamin B 12 analogs and panthenol, interferon agonists and antagonists, hydroxyacids such as those described in U.S. Pat. No. 5,550,158, benzophenones, and hydantoin anticonvulsants such as phenytoin, and combinations thereof. Other additional hair growth stimulants are described in JP 09-157,139 to Tsuji et al., published Jun. 17, 1997; EP 0277455 A1 to Mirabeau; WO 97/05887 to Cabo Soler et al.; WO 92/16186 to Bonte et al.; JP 62-93215 to Okazaki et al.; U.S. Pat. No. 4,987,150 to Kurono et al.; JP 290811 to Ohba et al., published Oct. 15, 1992; JP 05-286,835 to Tanaka et al., published Nov. 2, 1993, FR 2,723,313 to Greff, U.S. Pat. No. 5,015,470 to Gibson, U.S. Pat. Nos. 5,559,092, 5,536,751, 5,714,515, EP 0,319,991, EP 0,357,630, EP 0,573,253, JP 61-260010, U.S. Pat. Nos. 5,772,990, 5,053,410, and 4,761,401. In some embodiments, the additional ingredient is either minoxidil or finasteride.

In some embodiments, further non-limiting examples of optional ingredients include preservatives, perfumes or fragrances, cationic polymers, viscosity modifiers, coloring agents or dyes, conditioning agents, hair bleaching agents, thickeners, moisturizers, foam boosters, additional surfactants or nonionic cosurfactants, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, non-volatile solvents or diluents (water-soluble and water-insoluble), co-solvents or other additional solvents, and similar other materials.

### Methods of Preparation

In some embodiments, the nano- or micro-emulsion is formed by at least one high shear homogenization step. In some embodiments, the nano- or micro-emulsion is formed by at least one high pressure homogenization step.

In some embodiments, the nano- or micro-emulsion is formed by mixing a buffer solution with an oil mixture, wherein the buffer solution comprises rhamnolipids and mannosylerythritol lipids and the oil mixture comprises black cumin oil, rosemary oil and peppermint oil in a 2:1:1 ratio by mass. In some embodiments, the mixing occurs via high shear homogenization. In some embodiments, the high shear homogenization is performed at 5 k rpm. In some embodiments, the high shear homogenization is performed at 10 k rpm. In some embodiments, the high shear homogenization is performed at 15 k rpm. In some embodiments, the high shear homogenization is performed at 20 k rpm. In some embodiments, the high shear homogenization is performed at 25 k rpm. In some embodiments, the high shear homogenization is performed at 30 k rpm. In some embodiments, the high shear homogenization is performed at 5 k rpm or higher. In some embodiments, the high shear homogenization is performed at 10 k rpm or higher. In some embodiments, the high shear homogenization is performed at 15 k rpm or higher. In some embodiments, the high shear homogenization is performed at 20 k rpm or higher. In some embodiments, the high shear homogenization is performed at 25 k rpm or higher. In some embodiments, the high shear homogenization is performed at 30 k rpm or higher.

In some embodiments, the mixing occurs via high pressure homogenization. In some embodiments, the mixing occurs via high pressure homogenization at 20,000 psi. In some embodiments, the mixing occurs via high pressure homogenization at 25,000 psi. In some embodiments, the mixing occurs via high pressure homogenization at 30,000 psi. In some embodiments, the mixing occurs via high pressure homogenization at 35,000 psi. In some embodiments, the mixing occurs via high pressure homogenization at 40,000 psi. In some embodiments, the mixing occurs via high pressure homogenization at 20,000 psi or higher. In some embodiments, the mixing occurs via high pressure homogenization at 25,000 psi or higher. In some embodiments, the mixing occurs via high pressure homogenization at 30,000 psi or higher. In some embodiments, the mixing occurs via high pressure homogenization at 35,000 psi or higher. In some embodiments, the mixing occurs via high pressure homogenization at 40,000 psi or higher.

In some embodiments, the mixing occurs via high pressure homogenization once. In some embodiments, the mixing occurs via high pressure homogenization twice. In some embodiments, the mixing occurs via high pressure homogenization three times. In some embodiments, the mixing occurs via high pressure homogenization at least three times. In some embodiments, the mixing occurs via microfluidizer.

In some embodiments, the mixture occurs in a manner to minimize the formation of foam.

In some embodiments, the formation of the nano- or micro-emulsion occurs at room temperature. In some embodiments, the formation of the nano- or micro-emulsion occurs at an elevated temperature. In some embodiments, the formation of the nano- or micro-emulsion occurs at a reduced temperature. In some embodiments, there is a heating step. In some embodiments, the heating step is at 40° C.

### EXAMPLES

While the present disclosure has been described in terms of exemplary aspects, those skilled in the art will recognize that the present disclosure can be practiced with modifications in the spirit and scope of the appended claims. These examples and embodiments given above are merely illustrative and are not meant to be an exhaustive list of all possible designs, aspects, applications or modifications of the present disclosure. The following examples are provided to help illustrate a use of the compositions discussed herein. hair care composition described herein. It will be appreciated that other modifications of the concentrated hair care compositions described herein within the skill of those in the emulsion formulation art can be undertaken without departing from the spirit and scope of this disclosure. All parts, percentages, and ratios herein are by weight unless otherwise specified. Some components may come from suppliers as dilute solutions. The amount stated reflects the weight percent of the active material, unless otherwise specified.

### Example 1. Interfacial Tension (IT) Between Oils and Water and Composition of Microemulsions

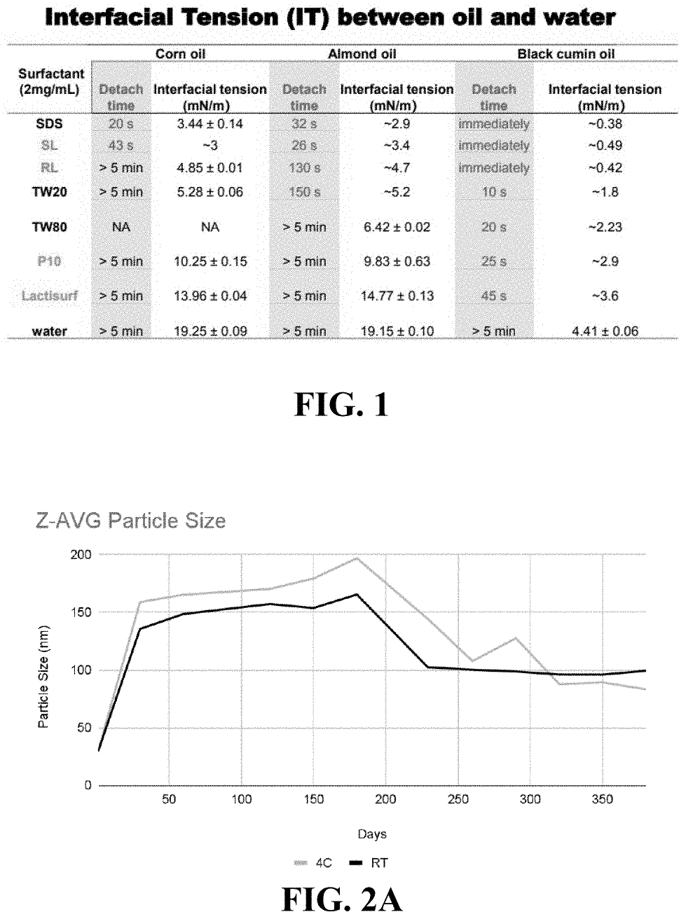

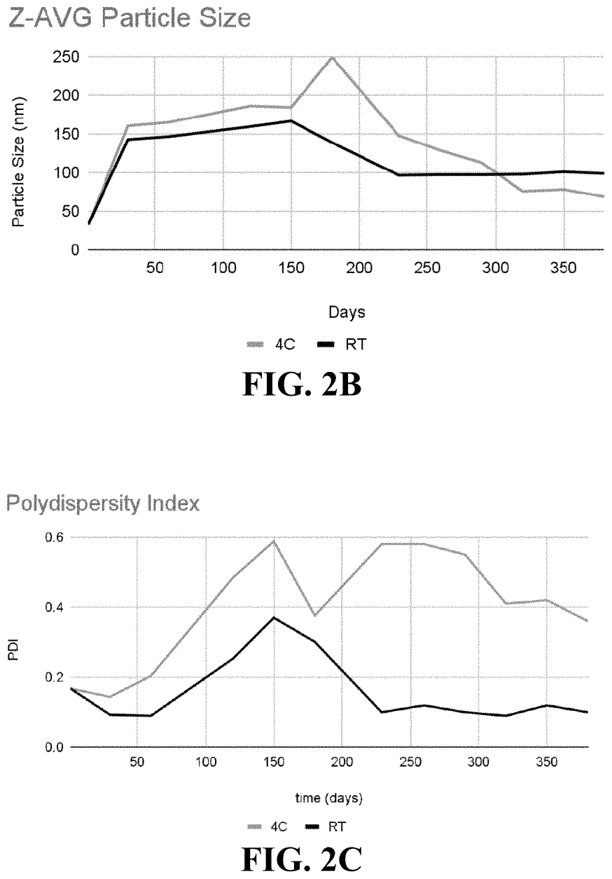

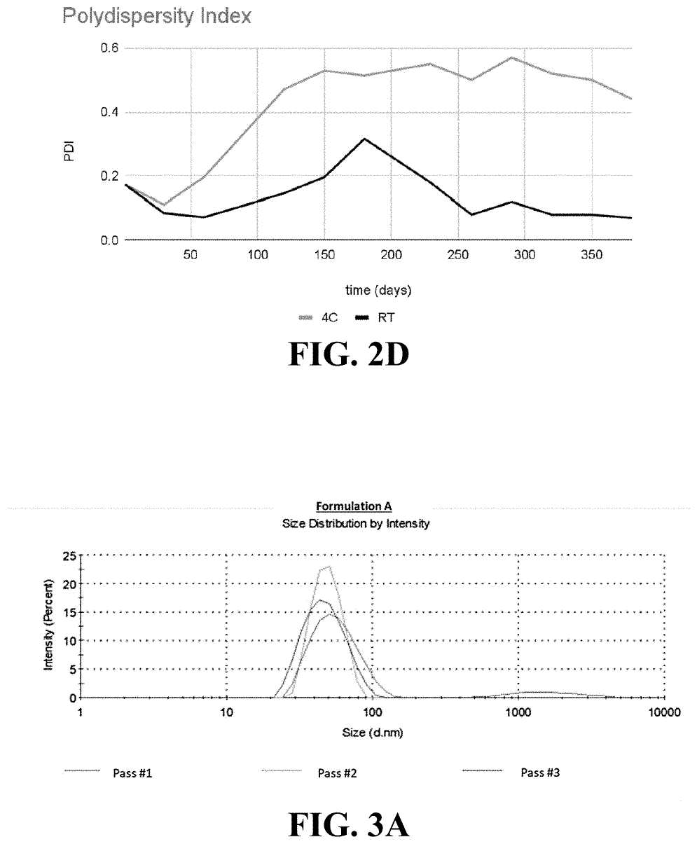

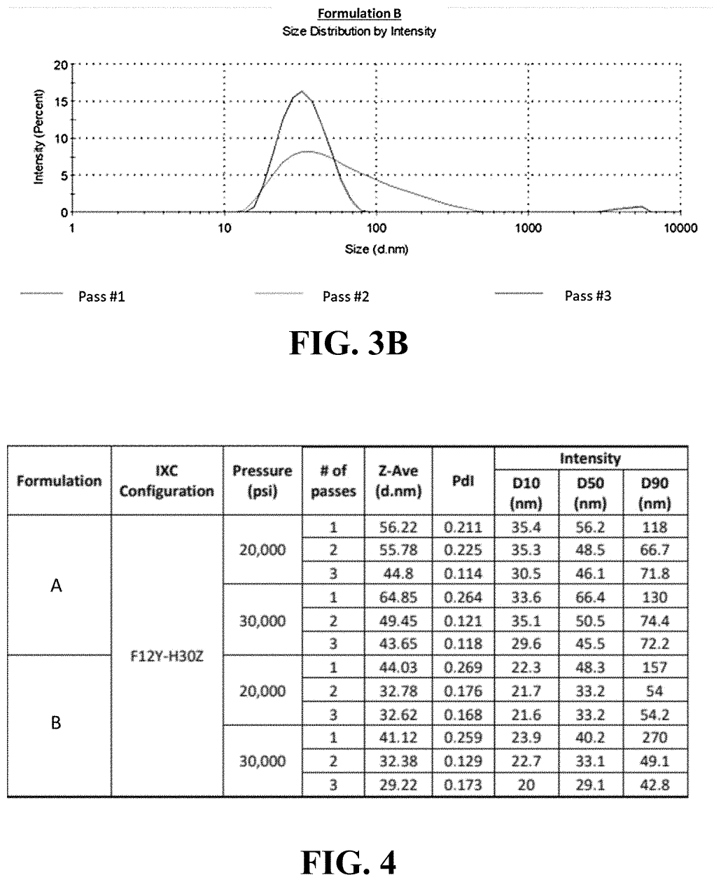

Various surfactants were examined with various oils to determine the detach time and interfacial tension (mN/m) with each combination of ingredients in preparation of the most successful combinations. The results can be seen in FIG. 1. These interfacial tension tests have shown that black cumin oil has surfactant qualities to it and may contain saponin FIG. 1. Without being bound to any theory, these findings suggest this saponin contributes to small particle size of these microemulsions and enhances stability. Stability of particle size and pdi are evaluated.

### Example Formulation 1

### 1% Black Cumin Oil

### 0.5% Peppermint Oil

### 0.5% Rosemary Oil

### 0.5% Mannosylerythritol Lipids

### 1% Rhamnolipids

### 1.25% Phosphatidylcholine

### 0.25% Salt

### 95% DI Water

20 k psi (microfluidizer)

The use of a buffer medium kept the formulations pH stable at around 7, and decreased the amount of foaming during the homogenization step of formulation. High amounts of black cumin oil (as well as essential oils) resulted in aggregation of emulsion droplets, so it was primarily used in low concentrations. Rhamnolipids were found to be a beneficial biosurfactant to this formulation, having the strongest impact on particle size and stability. Formulations A, B and C have shown excellent properties regarding particle size and polydispersity.

| Formulation A | | Formulation B | | Formulation C | |
|---|---|---|---|---|---|
| 1% | Black Cumin Oil | 1% | Black Cumin Oil | 1% | Black Cumin Oil |
| 0.5% | Peppermint Oil | 0.5% | Peppermint Oil | 0.5% | Peppermint Oil |
| 0.5% | Rosemary Oil | 0.5% | Rosemary Oil | 0.5% | Rosemary Oil |
| 0.25% Mannosylerythritol | | 0.25% Mannosylerythritol | | 0.25% Mannosylerythritol | |
| Lipids | | Lipids | | Lipids | |
| 1% | Rhamnolipids (50%) | 2% | Rhamnolipids (50%) | 5% | Rhamnolipids (50%) |
| 0.625% | Phosphatidylcholine | 0.625% | Phosphatidylcholine | 0.625% | Phosphatidylcholine |
| 92.125% | Buffer Solution | 91.125% | Buffer Solution | 88.125% | Buffer Solution |
| 2% | Lactobacillus ferment | 2% | Lactobacillus ferment | 2% | Lactobacillus ferment |
| 2% Cocos Nucifera Extract & | | 2% Cocos Nucifera Extract & | | 2% Cocos Nucifera Extract & | |
| Lactobacillus ferment | | Lactobacillus ferment | | Lactobacillus ferment | |
| 30k | psi (HPH) | 30k | psi (HPH) | 30k | psi (HPH) |

Each formulation was also tested at 20 k psi HPH where indicated.

### Example 2. Preparation of Microemulsion of Formulation A

Step 1. Buffer (1 L): add 1.549 g of Na₂HPO₄-7H₂O and 0.583 g of NaH₂PO₄-H₂O to a beaker. Add 500 mL distilled water and stir until fully dissolved. Adjust the solution to pH 7.0 with HCl or NaOH. Add water until the final volume is 1 L.

Step 2. Oil phase: in a beaker, measure 10 g of Black Cumin Oil, 5 g of Rosemary Oil, and 5 g of Peppermint Oil by mass. Stir at room temperature until dissolved.

Step 3. Aqueous phase: in a beaker, measure 2.5 g of Mannosylerythritol Lipids, 6.25 g of Phosphatidylcholine, and 20 g of Rhamnolipids by mass (Rhamnolipids is supplied as a 50% aqueous solution, 20 g of 50% RL is equivalent to 10 g of pure RL and 10 g of water). Add 910.25 g buffer (911.25 g buffer-1 g water from RL), 20 g SF Lactobacillus ferment, and 20 g of Cocos Nucifera Extract & Lactobacillus ferment. Magnetically stir at 40° C. until the surfactants disperse, and the solution is opaque (stirring too rapidly will cause foaming). Remove the stir bar and cool to room temperature.

Step 4. Coarse emulsion: add the oil phase to the aqueous phase. High shear homogenize at 10 k rpm for 4 minutes or until the emulsion has been formed.

Step 5. Fine emulsion: pour the coarse emulsion into the High-Pressure Homogenization (HPH) reservoir, avoiding any foam generated from high shear homogenization. Pass the entire volume of the coarse emulsion through the HPH at the desired pressure, then return the processed fine emulsion back to the reservoir for a total of 3 passes. This step can also be done with a microfluidizer.

Preparation of the other formulations proceeds in a similar manner with the amount of rhamnolipid adjusted accordingly.

### Example 3. Clinical Study to Assess the Efficacy of the Nano- or Micro-Emulsions of Formulations 1, A, B and C

The evaluation of the formulation involves tracking the stability of the particle size and polydispersity index. Additionally, the evaluation of the nano- or micro-emulsion involves hair follicle penetration testing as well as safety and irritation testing.

### Objective

Clinical study to assess the consumer perception and efficacy of a scalp treatment in improving hair appearance.

### Study Duration

The study is completed within a 6-month period.

### Study Design and Methods

The study evaluates Formulation 1, Formulation A, Formulation B and Formulation C.

### Use Instructions

The tested formulation is used twice per day (morning and night). One (1) dropperful (1 mL) is applied directly onto scalp where desired. Massage for 10 seconds into with scalp fingertips. Do not rinse.

### Selection Criteria

Panel selection is accomplished by advertisements in local periodicals, community bulletin boards, phone solicitation, electronic media or any combination thereof. Individuals are admitted to study at the discretion of the Investigator or his designee based on medical history and findings on the pre-study interview and examination.

Number of Subjects: Approximately 50 healthy subjects meeting inclusion/exclusion criteria listed below are enrolled to complete the study with a minimum of 30 subjects.

Age: 35-55

Sex: Male and Female

Race: Unrestricted

Skin Type: Unrestricted

### Inclusion Criteria

Individuals who, at baseline, are free of any dermatological or systemic disorder, which would interfere with the results.
Individuals in good general health.
Individuals who complete a preliminary medical history.
Individuals who read, understand and sign an informed consent document.
Individuals who are able to cooperate with the Investigator and research staff, have the test product applied according to the protocol and complete the full course of the study.
Individuals who have not participated in any study involving the same test site (hair/scalp) for the past 6 months
Individuals who agree to continue to use their current personal hair care products (e.g. shampoo, conditioner, mousse, hairspray) for the duration of the study and agree to not incorporate any new products with the exception of those provided by BCS.
Women with self-perceived thinning areas or overall thinning (diffuse thinning of the top of the crown, temples, widening part or receding hair line). +Confirmed by expert grader as I-4, I-2, I-3, I-4 on the Savin scale for hair part. Temples and overall thinning also confirmed by expert.
Men with self-perceived thinning areas or overall thinning (balding spot at the top of the head, visible hairline gap, receding hairline) of the hair. Confirmed by expert grader as II, III, III Vertex, IV, V, IIA on the Hamilton-Norwood Scale.
Individuals who agree to maintain the same length, hair styling and coloring practices for the duration of the study.
Females who agree to take a urine pregnancy test prior to enrollment.
Individuals who are not currently using and agree not to use for the duration of the study any other supplements or medications for hair loss and thinning, or those that affect hair growth.

### Exclusion Criteria:

Individuals who have had a history of any acute or chronic disease that could interfere with or increase the risk on study participation.
Individuals with an active (flaring) disease or chronic skin allergies (atopic dermatitis/eczema), or had recently treated skin cancer (within the last 12 months).
Individuals with damaged skin at or in close proximity to test sites (e.g., sunburn, tattoos, scars, excessive dandruff or other disfigurations).
Individuals having a health condition and/or pre-existing or dormant dermatologic disease on the scalp/hairline (e.g., psoriasis, rosacea, acne, eczema, seborrheic dermatitis, severe excoriations etc.) that the Investigator or designee deems inappropriate for participation or could interfere with the outcome of the study.
Individuals who have any history, which, in the Investigator's opinion, indicates the potential for harm to the subject or could place the validity of the study in jeopardy.
Individuals who indicate that they are pregnant, planning a pregnancy or nursing.
Individuals who have been medically diagnosed with Type I diabetes.
Individuals who have had any medical procedure, such hair replacement, hair plugs, or plastic surgery to the test site (scalp).
Individuals who have been diagnosed with alopecia (alopecia areata, scarring alopecia, traction alopecia, chemical alopecia, trichotillomania or trichorrhexis nodosa)
Individuals having severe hair loss or completely bald
Individuals who are currently using, or have used within 6 months of the study start, minoxidil, Rogaine, finasteride, or Propecia, or anti-androgen therapies (eg. Spironolactone) or any oral supplements designed to prevent hair loss or promote growth.
Currently participating in any other consumer perception study or clinical trial on the same test site (hair/scalp), another research facility, or doctor's office
Individuals who have recently (within the last 6 months) started the use of hormones for birth control or hormone replacement therapy (HRT). Those currently using hormones for birth control or HRT must have been on a stable dose (6 months or longer) in order to be eligible for the study (the initiation of HRT or birth control should not have been associated with the initiation of hair loss/thinning).
Participants who are using any topical treatment or washout (shampoo or conditioners) with claims on hair growth.
Individuals who have a known history of hypersensitivity to any cosmetics, personal care products, and/or fragrances.
Individuals who have used Low Level Laser therapy or platelet rich plasma within the last 3 months.
A known history of autoimmune thyroid disease, any other thyroid disorder/abnormality or other autoimmune disorders that are not controlled in the opinion of the investigator may interfere with the study treatment.
History of malignancy (except scc and bcc skin cancers) or undergoing chemotherapy or radiation treatments.

### Experimental Techniques

### Digital Photography for Expert Grading of Appearance of Hair Volume

Consistency of photographic results is achieved by eliminating all variables except the color and luminosity of the skin. Light source to subject distance is constant for all photos. The flash units are set to manual so the light output is the same for every picture. The camera is set to manual and the aperture is determined using a light meter. The aperture and shutter speed is held constant throughout the study. The images are not digitally enhanced.

Enrolled subjects will have photographs of three two (32) areas affected (each temple, balding spot, hairline, hair part, back of the head) of the hair/scalp obtained using a digital camera at each interval. Digital photographs are expert graded for appearance of hair volume using the following scale: (half-point increments are used)

Scale: 0=No visible hair volume; 1-3=Mild visible hair volume; 4-6=Moderate visible hair volume; 7-9=Severe hair volume

### Self-Assessment Questionnaire

Each subject is instructed to complete a self-assessment questionnaire at the baseline, immediate, 3 months, and 6 months post-treatment intervals.

### Procedure

1. Subjects will report to the facility at the start of the study with clean hair (no leave-on products applied).
2. Prior to beginning any study related activities, subjects are given an informed consent form, HIPAA form, code of conduct, and photography release form to read.
3. Once subject has completed reading, they are interviewed, in private, by BioScreen to ensure their understanding of the aforementioned forms and be given the opportunity to ask any study related questions.
4. Subjects who agree to sign the informed consent, HIPAA form, code of conduct, and photography release forms are asked to complete a medical history form. Subjects declining to sign the aforementioned forms are dismissed from the study.
5. Subjects are enrolled on the basis of the subject selection criteria. Subjects failing to meet criteria are dismissed from the study.
6. Enrolled subjects are given specific instructions continue usage of current personal hair care products (e.g. shampoo, conditioner, mousse, and hairspray) for the duration of the study and agree to not incorporate any new products with the exception of those provided by BCS.

### Baseline (Pre-Treatment)

Digital photography
Self-assessment questionnaire
Subjects are instructed to apply the test product according to the provided use instructions.
Following product application, the subjects are instructed to complete a self-assessment questionnaire.
Subjects are dismissed from the testing facility and instructed to return 3 months (3 days) and 6 months (±3 days) post-treatment. Subjects are provided with a list of parameters to keep in mind during use of the test products, which will assist them in completion of the post-treatment questionnaires accurately. Test products are weighed and recorded prior to dispensing to subjects. Subjects are instructed during the course of the study to maintain the same style, color, and length of hair as seen on the baseline visit.
Subjects are instructed to return to the testing facility with clean hair (no leave-in products applied). Test product is weighted for compliance. Subjects suspected of non-compliance are dismissed from study participation. Subjects are instructed not to use test product on the day of their scheduled visits until the study visit has completed.
Subjects will have the below procedures/measurements performed by trained BCS staff:
   3 Month (±0.3 days) 6 Month (±0.3 days) Post-treatment
Digital photography
Self-assessment questionnaire
Subjects return the remaining test products at 6 month visit and are dismissed from the study.

### Adverse Events

An adverse event is any untoward medical occurrence, whether or not it is considered study related, including death, experienced by a subject. An event may consist of a disease, an exacerbation of a pre-existing illness or condition, an occurrence of an intermittent illness or condition, a set of related symptoms or signs, or a single symptom or sign.

A serious adverse event (SAE) as defined in the CFR 312.32 is “any experience that is fatal or life threatening, is permanently disabling, requires inpatient hospitalization, or is a congenital anomaly, cancer, or overdose”. All serious adverse events is reported within 24 hours of BCS notification.

Each adverse event must be promptly recorded and sufficiently documented by the Study Director in the source documentation and case report form even if the adverse event is assessed by the Study Director as unlikely to be related to the study. Adverse events are graded on a scale of severity (mild, moderate, severe, or life-threatening) and on a scale of relationship to the product (unknown, unrelated, unlikely, possible, probable, or definite). All adverse events are reported within five business days. All adverse events are followed up until resolved, stabilized, the subject is lost to follow-up or the event is otherwise explained. All follow-up information should be reported.

If, according to the Investigator, medical care is warranted, appropriate referrals are made. BCS will follow all adverse events until resolution.

### Risks/Discomforts

Potential risks/discomforts include the possibility of slight irritation, itching, stinging, and/or burning. There also may be risks and discomforts, which are not yet known.

### Subject Discontitmation

Criteria for the discontinuation of a subject during the study will include the following:
Significant protocol violation
Serious adverse experience
Request of the subject
Any unmanageable factor, in the Investigator's opinion, that may significantly interfere with the protocol or interpretation of results.

### Protocol Amendment

Any changes to the study protocol are approved in writing by the client and BCS prior to implementation in the study.

Data Analyses

Statistical analyses will test the hypothesis that the pre-treatment values of each parameter are statistically different from its post-treatment values. In the case of a normal distribution the Student's paired t-test are employed and in the case of not normal distribution, the Wilcoxon Signed Rank Test is employed. Statistical significance is declared if the two-tailed p-value is ≤0.05.

Subject scores for each parameter in questionnaires is presented in a tabular format. The percentage of subjects responding in favor of the test product is reported. Statistical analysis is performed using a z-test. Statistical significance is declared if the p-value is ≤0.05.

### Study Report

Interim report is issued within 10 business days of Month 3 interval completion. Final report is issued within four weeks of study completion.

### Example 4. Franz Cell Testing

These skin absorption assays evaluate linoleic acid penetration using human ex vivo skin explants. The procedure involves treating 15 frozen human skin explants as follows:
Control condition: non-treated, 3 individual explant of 2 cm² non treated
Skin treated with linoleic acid: 6 individual explants of 2 cm² treated with linoleic acid
Skin treated with formulation containing linoleic acid: 6 individual explants of 2 cm² treated with formulation

Skin explants are treated (Franz cells) following the protocols outlined in OCDE No 428 Guidelines (skin absorption: in vitro method) (which is incorporated by reference in its entirety) using frozen skin explants.

Each formulation is to be applied at 250 mg/cm² estimated on linoleic acid passage efficacy and detection limit. After formulation treatment during 24 h incubation time, following steps are be performed:
Compound that went through the skin will be collected in a container (50% water-50% EtOH or PBS (pH 7.4)+0.1% cc.
Remaining compound on skin surface will be collected
Compound present in stratum corneum will be collected: multiple application of 2 strips (D-squam) collected in a vial
Compound present in epidermis and dermis layers will be collected: skin explants will be placed on a heating plate. Epidermis and dermis layers will be separated and placed in separate vials
   Quantify, in a first step, compound present in different skin zones:

1. Epidermis

2. Dermis

Other compartments will be kept frozen for analysis in a second step, according to first results.

### Example 5. Acute Primary Skin Patch Test

To determine the primary (acute) irritation potential of a test material after a single application to the skin of human subjects for 48 hours.

### Inclusion Criteria:

Individuals who were not currently under a doctor's care.

Individuals who were free of any dermatological or systemic disorder that would interfere with the results, at the discretion of the Investigator.

Individuals who were free of any acute or chronic disease that would interfere with or increase the risk on study participation.

Individuals who completed a preliminary medical history form mandated by BCS and were in general good health.

Individuals who read, understood and signed an informed consent document relating to the specific type of study.

Individuals who were able to cooperate with the Investigator and research staff, and were willing to have test materials applied according to the protocol, and complete the full course of the study.

### Exclusion Criteria:

Individuals who were under 18 years of age.

Individuals who were currently under a doctor's care. Individuals who were currently taking any medication (topical or systemic) that might mask or interfere with the test results.

Individuals who had a history of any acute or chronic disease that might interfere with or increase the risk associated with study participation.

Individuals who were diagnosed with chronic skin allergies.

Female volunteers who indicated that they were pregnant or nursing.

### Population Demographics:

Number of subjects enrolled 55
Number of subjects completing study 55

### Age Range 18-61

### Sex

### Male 12

### Female 43

### Fitzpatrick Skin Type*

1—always burn, does not tan 0
2—burn easily, tan slightly 3
3—burn moderately, tan progressively 19
4—burn a little, always tan 21
5—rarely burn, tan intensely 12
6—never burn, tan very intensely 0

### Test Material Handling.

Test materials to be tested under occlusive conditions were placed on an 8-millimeter aluminum Finn Chamber® (Epitest Ltd. Oy, Tuusula, Finland) supported on Scanpor® Tape (Norgesplaster A/S, Kristiansand, Norway) or an 8-millimeter filter paper coated aluminum Finn Chamber® AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRA® closed cell system which is made of additive-free polyethylene plastic foam with a filter paper incorporated (It is supplied in units of 10 chambers on a hypoallergenic non woven adhesive tape; the width of the tape is 52 mm and the length is 118 mm) or other equivalents.

Test materials to be tested under semi-occlusive conditions were placed on a test strip with a Rayon/Polypropylene pad or on a 7.5 mm filter paper disc affixed to a strip of hypoallergenic tape (Johnson & Johnson 1 inch First Aid Cloth Tape).

Test materials to be tested in an open-patch were rubbed directly onto skin for approximately one (1) minute.

Approximately 0.02-0.05 mL (in case of liquids) and/or 0.02-0.05 gm (in case of solids) of the test material was used for the study. Liquid test material was dispensed on a 7.5 mm paper disk, which fit in the Finn Chamber.

### Procedure

Subjects were requested to bathe or wash as usual before arrival at the facility.

Patches containing the test material were then affixed directly to the skin of the intrascapular regions of the back, to the right or left of the midline and subjects were dismissed with instructions not to wet or expose the test area to direct sunlight.

Patches will remain in place for 48 hours. Subjects were instructed not to remove the patches prior to their next scheduled visit.

Trained skin grading laboratory personnel removed the patch and evaluated the test sites.

In the event of an adverse reaction, the area of erythema and edema is measured. The edema is estimated by the evaluation of the skin with respect to the contour of the unaffected normal skin.

Scoring scale and definition of symbols shown below are based on the scoring scheme according to the International Contact Dermatitis Research Group scoring scale [Rietschel, R. L., Fowler, J. F., Ed., Fisher's Contact Dermatitis (fourth ed.). Baltimore, Williams & Wilkins, 1995] listed below:

| | |
|---|---|
| 0 | no reaction (negative) |
| 1 | erythema throughout at least ¾ of patch area |
| 2 | erythema and induration throughout at least ¾ of patch area |
| 3 | erythema, induration and vesicles |
| 4 | erythema, induration and bullae |
| D | Site discontinued |
| Dc | Subject discontinued |
| DcI | Subject discontinued per Investigator |

Clinical evaluations are performed by a BCS investigator or designee trained in the clinical evaluation of the skin. Whenever feasible, the same individual will do the scoring of all the subjects throughout the study and is blinded to the treatment assignments and any previous scores.

### Results

Test Material Description: micro- or nano-emulsion Formulation A
micro- or nano-emulsion Formulation B

### Patch Description: Semi-Occlusive

| Identifiers | | | | Response to | | Response to | |
|---|---|---|---|---|---|---|---|
| | | | Skin | Formulation A | | Formulation B | |
| No. | Sex | Age | Type | 0 HR | 48 HRS | 0 HR | 48 HRS |
| 1 | F | 38 | 4 | 0 | 0 | 0 | 0 |
| 2 | M | 59 | 4 | 0 | 0 | 0 | 0 |
| 3 | M | 39 | 4 | 0 | 0 | 0 | 0 |
| 4 | F | 29 | 5 | 0 | 0 | 0 | 0 |
| 5 | M | 18 | 3 | 0 | 0 | 0 | 0 |
| 6 | M | 29 | 4 | 0 | 0 | 0 | 0 |
| 7 | F | 40 | 4 | 0 | 0 | 0 | 0 |
| 8 | F | 49 | 5 | 0 | 0 | 0 | 0 |
| 9 | F | 38 | 5 | 0 | 0 | 0 | 0 |
| 10 | F | 38 | 4 | 0 | 0 | 0 | 0 |
| 11 | F | 25 | 4 | 0 | 0 | 0 | 0 |
| 12 | F | 27 | 4 | 0 | 0 | 0 | 0 |
| 13 | F | 23 | 3 | 0 | 0 | 0 | 0 |
| 14 | F | 28 | 4 | 0 | 0 | 0 | 0 |
| 15 | F | 31 | 4 | 0 | 0 | 0 | 0 |
| 16 | F | 34 | 2 | 0 | 0 | 0 | 0 |
| 17 | F | 31 | 2 | 0 | 0 | 0 | 0 |
| 18 | M | 57 | 3 | 0 | 0 | 0 | 0 |
| 19 | M | 29 | 5 | 0 | 0 | 0 | 0 |
| 20 | F | 34 | 3 | 0 | 0 | 0 | 0 |
| 21 | F | 23 | 4 | 0 | 0 | 0 | 0 |
| 22 | M | 26 | 4 | 0 | 0 | 0 | 0 |
| 23 | F | 26 | 5 | 0 | 0 | 0 | 0 |
| 24 | F | 58 | 3 | 0 | 0 | 0 | 0 |
| 25 | F | 20 | 4 | 0 | 0 | 0 | 0 |
| 26 | F | 22 | 3 | 0 | 0 | 0 | 0 |
| 27 | F | 41 | 5 | 0 | 0 | 0 | 0 |
| 28 | F | 34 | 5 | 0 | 0 | 0 | 0 |
| 29 | F | 35 | 3 | 0 | 0 | 0 | 0 |
| 30 | F | 25 | 5 | 0 | 0 | 0 | 0 |
| 31 | F | 42 | 5 | 0 | 0 | 0 | 0 |
| 32 | M | 35 | 3 | 0 | 0 | 0 | 0 |
| 33 | F | 31 | 4 | 0 | 0 | 0 | 0 |
| 34 | F | 46 | 3 | 0 | 0 | 0 | 0 |
| 35 | F | 61 | 3 | 0 | 0 | 0 | 0 |
| 36 | F | 57 | 3 | 0 | 0 | 0 | 0 |
| 37 | F | 56 | 4 | 0 | 0 | 0 | 0 |
| 38 | F | 32 | 5 | 0 | 0 | 0 | 0 |
| 39 | F | 42 | 4 | 0 | 0 | 0 | 0 |
| 40 | F | 54 | 3 | 0 | 0 | 0 | 0 |
| 41 | F | 19 | 4 | 0 | 0 | 0 | 0 |
| 42 | F | 23 | 3 | 0 | 0 | 0 | 0 |
| 43 | M | 29 | 3 | 0 | 0 | 0 | 0 |
| 44 | M | 39 | 3 | 0 | 0 | 0 | 0 |
| 45 | F | 35 | 2 | 0 | 0 | 0 | 0 |
| 46 | M | 35 | 4 | 0 | 0 | 0 | 0 |
| 47 | F | 37 | 3 | 0 | 0 | 0 | 0 |
| 48 | F | 31 | 4 | 0 | 0 | 0 | 0 |
| 49 | F | 41 | 3 | 0 | 0 | 0 | 0 |
| 50 | M | 55 | 4 | 0 | 0 | 0 | 0 |
| 51 | F | 18 | 5 | 0 | 0 | 0 | 0 |
| 52 | F | 57 | 3 | 0 | 0 | 0 | 0 |
| 53 | F | 54 | 5 | 0 | 0 | 0 | 0 |
| 54 | F | 36 | 4 | 0 | 0 | 0 | 0 |
| 55 | F | 42 | 3 | 0 | 0 | 0 | 0 |

### Observation:

No adverse reactions of any kind were reported during the course of this study.

There were sixteen (16) subjects with a Grade 1 reaction to the positive control (2.0% Sodium Lauryl Sulfate Solution).

No subjects showed any signs of reaction to the negative control (DI Water).

### CONCLUSIONS

Under the conditions of the study, there were no indications of a potential to elicit dermal irritation for the micro- or nano-emulsion Formulation A and micro- or nano-emulsion Formulation B.

### REFERENCES

Ohadi, M., Shahravan, A., Dehghan, N., Eslaminejad, T., Banat, I. M., & Dehghannoudeh, G. (2020). Potential Use of Microbial Surfactant in Microemulsion Drug Delivery System: A Systematic Review. Drug Design, Development and Therapy, 14, 541-550.
Yukuyama, M. N., Ghisleni, D. D. M., Pinto, T. J. A., & Bou-Chacra, N. A. (2015). Nanoemulsion: process selection and application in cosmetics—a review. International Journal of Cosmetic Science, 38 (1), 13-24.
Akter, N., Radiman, S., Mohamed, F., & Reza, M. (2013). Self-Assembled Potential Bio Nanocarriers for Drug Delivery. Mini-Reviews in Medicinal Chemistry, 13 (9), 1327-1339
Lopes, L. (2014). Overcoming the Cutaneous Barrier with Microemulsions. Pharmaceutics, 6 (1), 52-77.
Gibaud, S., & Attivi, D. (2012). Microemulsions for oral administration and their therapeutic applications. Expert Opinion on Drug Delivery, 9 (8), 937-951.
Bandier J, Carlsen B C, Rasmussen M A, Petersen L J, Johansen J D. (2015) Skin reaction and regeneration after single sodium lauryl sulfate exposure stratified by filaggrin genotype and atopic dermatitis phenotype. Br J Dermatol. 172 (6):1519-1529.
Rossi, A., Priolo, L., Iorio, A., Vescarelli, E., Gerardi, M., Campo, D., . . . Marchese, C. (2013). Evaluation of a Therapeutic Alternative for Telogen Effluvium: A Pilot Study. Journal of Cosmetics, Dermatological Sciences and Applications, 03 (03), 9-16.
Panahi, Y., Taghizadeh, M., Marzony, E. T., & Sahebkar, A. (2015). Rosemary oil vs minoxidil 2% for the treatment of androgenetic alopecia: a randomized comparative trial. Skinmed, 13 (1), 15-21.
Murata, K., Noguchi, K., Kondo, M., Onishi, M., Watanabe, N., Okamura, K., & Matsuda, H. (2013). Promotion of hair growth by Rosmarinus officinalis leaf extract. Phytotherapy research: PTR, 27 (2), 212-217.
Oh, J. Y., Park, M. A., & Kim, Y. C. (2014). Peppermint Oil Promotes Hair Growth without Toxic Signs. Toxicological Research, 30 (4), 297-304.
Knorr, F., Lademann, J., Patzelt, A., Sterry, W., Blume-Peytavi, U., & Vogt, A. (2009). Follicular transport route—Research progress and future perspectives. European Journal of Pharmaceutics and Biopharmaceutics, 71 (2), 173-180.
Jiang, Q., Wu, Y., Zhang, H., Liu, P., Yao, J., Yao, P., Chen, J., & Duan, J. (2017). Development of essential oils as skin permeation enhancers: penetration enhancement effect and mechanism of action. Pharmaceutical biology, 55 (1), 1592-1600.
Azeem, A., Rizwan, M., Ahmad, F., Khan, Z., Khar, R., Aqil, M., & Talegaonkar, S. (2008). Emerging Role of Microemulsions in Cosmetics. Recent Patents on Drug Delivery & Formulation, 2 (3), 275-289.
Fracchia, Letizia & Banat, Jared & Cavallo, Massimo & Ceresa, Chiara & Banat, Ibrahim. (2015). Potential therapeutic applications of microbial surface-active compounds. AIMS Bioengineering. 2. 144-162.
Guo, Y., Mao, X., Zhang, J., Sun, P., Wang, H., Zhang, Y., Ma, Y., Xu, S., Lv, R., & Liu, X. (2019). Oral delivery of lycopene-loaded microemulsion for brain-targeting: preparation, characterization, pharmacokinetic evaluation and tissue distribution. Drug delivery, 26 (1), 1191-1205.
Ahmad, A., Husain, A., Mujeeb, M., Khan, S. A., Najmi, A. K., Siddique, N. A., Damanhouri, Z. A., Anwar, F. A review on therapeutic potential of Nigella sativa: A miracle herb. Asian Pac. J. Trop. Biomed., 3, 337-352 (2013).
U. Saleem, S. Sabir, B. Ahmad. Protective role of Nigella sativa in chemotherapy-induced alopecia Bangladesh J. Pharmacol. 12, 455-462 (2017)
Dell'Acqua G, Richards A. Black Cumin Seed Oil protects human hair follicle dermal papilla cells from stress induced growth inhibition and restores cell growth. In proceedings 31st IFSCC Congress, Yokohama, Japan, October 20-23, 2020.
Wińska, K., M4czka, W., Lyczko, J., Grabarczyk, M., Czubaszek, A., & Szumny, A. Essential Oils as Antimicrobial Agents-Myth or Real Alternative? Molecules (Basel, Switzerland), 24(11), 2130, 2019.
Morita T, Fukuoka T, Imura T, Kitamoto D. Production of mannosylerythritol lipids and their application in cosmetics. Appl Microbiol Biotechnol. 97(11):4691-700, 2013.
Coelho A L S, Feuser P E, Carciofi B A M, de Andrade C J, de Oliveira D. Mannosylerythritol lipids: antimicrobial and biomedical properties. Appl Microbiol Biolechnol. 104(6):2297-2318, 2020.
Chen J, Wu Q, Hua Y, Chen J, Zhang H, Wang H. Potential applications of biosurfactant rhamnolipids in agriculture and biomedicine. Appl Microbiol Biotechnol. 101(23-24):8309-8319, 2017.
Furse S, de Kroon A I. Phosphatidylcholine's functions beyond that of a membrane brick. Mol Membr Biol. 32(4):117-9, 2015.
21 CFR. Ch.1. Part 50, Subpart B.
Arch. Dermatol., 128: 347-351, 1992.
Br. J. Dermatol., 130: 167-173, 1994.
Skin Pharmacol. Appl. Skin Physiol., 16: 100-107, 2003
Pavoni L, Perinelli D R, Bonacucina G, Cespi M, Palmieri G F. An Overview of Micro- and Nanoemulsions as Vehicles for Essential Oils: Formulation, Preparation and Stability Nanomaterials (Basel). 10(1):135, 2020
Nastiti CMRR, Ponto T, Abd E, Grice J E, Benson H A E, Roberts M S. Topical Nano and Microemulsions for Skin Delivery. Pharmaceutics. 9(4):37, 2017
Fonseca-Santos B, Gremiao M P, Chorilli M. Nanotechnology-based drug delivery systems for the treatment of Alzheimer's disease. Int J Nanomedicine. 10:4981-5003, 2015
Roberts M S, Mohammed Y, Pastore M N, Namjoshi S, Yousef S, Alinaghi A, Haridass I N, Abd E, Leite-Silva V R, Benson H, Grice J E. Topical and cutaneous delivery using nanosystems. J Control Release 247:86-105, 2017

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

What is claimed is:

1. An oil-in-water nano- or micro-emulsion comprising black cumin oil and one or more rhamnolipid.

2. A nano- or micro-emulsion composition comprising: (a) an oil dispersion comprising black cumin oil, and (b) an aqueous solution comprising a rhamnolipid.

3. The nano- or micro-emulsion composition of claim 1 or 2, wherein the oil dispersion further comprises one or more of the group consisting of rosemary oil, peppermint oil, lavender oil, bergamot oil, lemon oil, orange, sandalwood oil, tea tree oil, chamomile oil, cedarwood oil, clary sage oil, lemongrass oil, carrot seed oil, and geranium oil.

4. The nano- or micro-emulsion composition of claims 1-3, wherein the oil dispersion further comprises rosemary oil, peppermint oil, or both.

5. The nano- or micro-emulsion composition of claims 1-4, wherein the aqueous solution comprises one or more of a mannosylerythritol lipid, phosphatidylcholine, and a preservative.

6. The nano- or micro-emulsion composition of claims 1-5, wherein the aqueous solution comprises a mannosylerythritol lipid, phosphatidylcholine, and a preservative.

7. The nano- or micro-emulsion composition of claims 1-6, comprising a preservative, wherein the preservative is one or more of Lactobacillus ferment and Lactobacillus Cocos Nucifera Fruit Extract.

8. The nano- or micro-emulsion composition of claims 1-7, wherein the composition comprises a preservative and the preservative is a combination of Lactobacillus ferment and Lactobacillus Cocos Nucifera Fruit Extract.

9. The nano- or micro-emulsion composition of claims 1-8, wherein the black cumin oil is present in 0.1% wt/wt to 5% wt/wt.

10. The nano- or micro-emulsion composition of claims 1-9, wherein the black cumin oil is present in about 1% wt/wt.

11. The nano- or micro-emulsion composition of claims 1-10, wherein the rhamnolipid is present in 1% wt/wt to 5% wt/wt.

12. The nano- or micro-emulsion composition of claim 1-11, wherein the rhamnolipid is present in about 1% wt/wt.

13. The nano- or micro-emulsion composition of claims 1-11, wherein the rhamnolipid is present in about 2% wt/wt.

14. The nano- or micro-emulsion composition of claims 1-11, wherein the rhamnolipid is present in about 5% wt/wt.

15. The nano- or micro-emulsion composition of claims 1-14, wherein the composition comprises black cumin oil and peppermint oil in a 1:1 ratio by weight, a 2:1 ratio by weight, or a 4:1 ratio by weight.

16. The nano- or micro-emulsion composition of claims 1-15, wherein the composition comprises black cumin oil and rosemary oil in a 1:1 ratio by weight, a 2:1 ratio by weight, or a 4:1 ratio by weight.

17. The nano- or micro-emulsion composition of claims 1-16, wherein the composition comprises black cumin oil, peppermint oil, and rosemary oil in a 1:1:1 ratio by weight, a 2:1:1 ratio by weight, a 1:2:1 ratio by weight, a 1:1:2 ratio by weight, a 2:2:1 ratio by weight, a 2:1:2 ratio by weight, a 1:2:2 ratio by weight, a 3:1:1 ratio by weight, a 1:3:1 ratio by weight, a 1:1:3 ratio by weight, a 4:1:1 ratio by weight, or a 5:1:1 ratio by weight.

18. The nano- or micro-emulsion composition of claims 1-17, wherein the composition comprises:
0.1-5% wt/wt black cumin oil;
0.1-5% wt/wt peppermint oil, and
0.1-5% wt/wt rosemary oil.

19. The nano- or micro-emulsion composition of claims 1-18, wherein the composition comprises:
0.1-5% wt/wt black cumin oil;
0.1-5% wt/wt peppermint oil;
0.1-5% wt/wt rosemary oil;
0.1-1% wt/wt mannosylerythritol lipids;
1-5% wt/wt rhamnolipids; and
0.1-2% wt/wt phosphatidylcholine.

20. The nano- or micro-emulsion composition of claims 1-19, wherein the oil dispersion particle size is less than 500 nm, less than 400 nm, less than 300 nm, or less than 250 nm.

21. The nano- or micro-emulsion composition of claims 1-20, wherein the oil dispersion particle size is less than 500 nm for 1 year or more.

22. The nano- or micro-emulsion composition of claims 1-21, wherein the oil dispersion particle size is less than 250 nm.

23. The nano- or micro-emulsion composition of claims 1-22, wherein the oil dispersion particle size is less than 250 nm for 1 year or more.

24. The nano- or micro-emulsion composition of claims 1-23, wherein the oil dispersion polydispersity index (Pdi) less than about 0.15.

25. The nano- or micro-emulsion composition of claims 1-24, wherein the oil dispersion polydispersity index is less than 0.15 for 90 days or more.

26. The nano- or micro-emulsion composition of claims 1-25, wherein the composition comprises 80-95% wt/wt of a buffer solution and 0.1-10% wt/wt of a preservative.

27. The nano- or micro-emulsion composition of claims 1-26, wherein the composition comprises 2% wt/wt Lactobacillus ferment; and 2% wt/wt Lactobacillus Cocos Nucifera Fruit Extract.

28. The nano- or micro-emulsion composition of claims 1-27, wherein the composition comprises:
1% wt/wt black cumin oil;
0.5% wt/wt peppermint oil;
0.5% wt/wt rosemary oil;
0.25% wt/wt mannosylerythritol lipids;
1%-5% wt/wt rhamnolipids;
0.625% wt/wt phosphatidylcholine;
88.125% wt/wt-92.125% wt/wt buffer solution;
2% wt/wt Lactobacillus ferment; and
2% wt/wt Lactobacillus/Cocos Nucifera Fruit Extract.

29. The nano- or micro-emulsion composition of claims 1-28, wherein the composition comprises an additional active pharmaceutical ingredient for the prevention of hair loss, reversing hair loss, and/or stimulating hair growth.

30. The nano- or micro-emulsion composition of claims 1-29 for use in the preparation of a medicament for the treatment of hair loss.

31. The nano- or micro-emulsion composition of claims 1-29 for use as a delivery vehicle for improving the bioavailability of other active ingredient(s).

32. A method for preventing hair loss, reversing hair loss, treating hair loss, stopping hair loss, or stimulating hair growth in a subject, comprising administering the nano- or micro-emulsion composition of claims 1-28.

33. The method of claim 32, wherein the nano- or micro-emulsion composition is topically administered or ingested.

34. The method of claim 32, wherein the nano- or micro-emulsion composition is administered topically.

35. The method of claim 32, wherein the nano- or micro-emulsion composition is ingested.

36. A method for improving bioavailability of an active pharmaceutical ingredient comprising adding the active pharmaceutical ingredient to the nano- or micro-emulsion composition of claims 1-31 and administering to a subject in need thereof.

37. The method of claim 36, wherein the nano- or micro-emulsion composition is topically administered or ingested.

38. The method of claim 36, wherein the nano- or micro-emulsion composition is administered topically.

39. The method of claim 36, wherein the nano- or micro-emulsion composition is ingested.

40. A method for preparing a nano- or micro-emulsion composition for administration to a subject comprising:
(a) preparing an oil phase comprising black cumin oil;
(b) preparing an aqueous phase comprising a rhamnolipid;
(c) homogenizing the oil phase and the aqueous phase; and
(d) obtaining the nano- or micro-emulsion comprising an oil dispersion.

41. The method of claim 40, wherein the oil dispersion further comprises one or more of rosemary oil or peppermint oil.

42. The method of claims 40-41, wherein the aqueous phase further comprises one or more of a mannosylerythritol lipid, phosphatidylcholine, Lactobacillus ferment and Lactobacillus Cocos Nucifera Fruit Extract.

43. The method of claims 40-42, wherein the oil dispersion further comprises rosemary oil and peppermint oil.

44. The method of claims 40-43, wherein the aqueous phase further comprises a mannosylerythritol lipid, phosphatidylcholine, Lactobacillus ferment and Lactobacillus Cocos Nucifera Fruit Extract.

45. The method of claims 40-44, wherein the black cumin oil is present in 0.5% wt/wt to 1.5% wt/wt.

46. The method of claims 40-45, wherein the black cumin oil is present in about 1% wt/wt.

47. The method of claims 40-46, wherein the rhamnolipid is present in 1% to 5% wt/wt.

48. The method of claim 40-47, wherein the rhamnolipid is present in about 1% wt/wt.

49. The method of claims 40-47, wherein the rhamnolipid is present in about 2% wt/wt.

50. The method of claims 40-47, wherein the rhamnolipid is present in about 5% wt/wt.

51. The method of claims 40-50, wherein the oil dispersion particle size is less than 200 nm.

52. The method of claims 40-51, wherein the oil dispersion particle size is less than 200 nm for 90 days or more.

53. The method of claims 40-52, wherein the oil dispersion polydispersity index (Pdi) less than 0.15.

54. The method of claims 40-53, wherein the oil dispersion polydispersity index is less than 0.15 for 90 days or more.

55. The method of claims 40-54, wherein the aqueous phase further comprises a buffered solution of pH between about 4 and about 8.

56. The method of claims 40-55, wherein the homogenizing step comprises high shear homogenization.

57. The method of claims 40-56, wherein the homogenizing step comprises high shear homogenization and high-pressure homogenization.

58. The method of claims 40-57, wherein the homogenizing step comprises high shear homogenization followed by high-pressure homogenization.

59. The method of claims 40-58, wherein the homogenizing step comprises performing high-pressure homogenization multiple times.

60. The method of claim 40-59, The method of claims 21-39, wherein the homogenizing step comprises performing high-pressure homogenization at least three times.

61. The method of claim 40-60, wherein the homogenizing step comprises performing high-pressure homogenization three times.

62. The method of claims 40-61, wherein the high shear homogenization is performed between 10 k rpm and 20 k rpm.

63. The method of claims 40-62, wherein the high-pressure homogenization is performed between 10 k psi and 30 k psi.

64. The method of claims 40-63, wherein the high-pressure homogenization is performed at 30 k psi.

65. The method of claims 40-64, wherein the method further comprises minimizing the formation of foam.

66. The composition according to any one of the preceding claims, wherein the composition comprises a buffer and wherein the buffer comprises an aqueous buffer.

67. The composition according to claim 66, wherein the aqueous buffer comprises sodium phosphate.

68. The composition according to claim 66, wherein the aqueous buffer comprises monosodium phosphate.

69. The composition according to claim 66, wherein the aqueous buffer comprises disodium phosphate.

70. The composition according to any one of the preceding claims, wherein the composition comprises one or more of lavender oil, bergamot oil, lemon oil, orange, sandalwood oil, tea tree oil, chamomile oil, cedarwood oil, clary sage oil, lemongrass oil, carrot seed oil, and geranium oil.
